(19) United States
(12) Patent Application Publication
(10) Pub. No.: US 2026/0335711 A1
(43) Pub. Date: Oct. 29, 2026

(54) Kinematic Analysis of Events During Playing of Sports Including Feedback Display

(71) Applicant: CONNEXA SPORTS TECHNOLOGIES INC., Windsor Mill, MD (US)

(72) Inventors: James Melvin PRIYARAJAN, Bangalore (IN); Tawfiq ALTAI, NSW (AU)

(21) Appl. No.: 19/112,421

(22) PCT Filed: Sep. 11, 2023
(86) PCT No.: PCT/IL2023/050979

§ 371 Date: Mar. 17, 2025

(30) Foreign Application Priority Data
Sep. 18, 2022 (IL) 296594

(51) Int. Cl. A63B 24/00; G06V 20/40

(52) U.S. Cl. CPC A63B 24/0006; G06V 20/44; A63B 2024/0009

(57) ABSTRACT

A method of performing kinematic analysis of a ball-striking event includes: receiving a video of a player during a ball-striking event containing a plurality of ball strikes; performing pose estimation on images of the player in multiple frames within the video, and deriving pose points of the player in each frame; determining a series of frames within the video in which a ball-strike occurs, for each series of frames in which a ball-strike occurs, orienting a view of the pose points in order to enable visual assessment of one or more execution parameters associated with kinematic performance of the player during one or more ball strikes within the ball-striking event; and displaying a visual representation of the one or more execution parameters overlaid on the oriented view of the pose points during the ball-striking event.

### RELATED APPLICATIONS

This Application claims the benefit of priority of Israeli Patent Application No. 296,594, filed Sep. 18, 2022, entitled “Kinematic Analysis of Events During Playing of Sports Including Feedback Display,” the contents of which are incorporated by reference as if fully set forth herein.

### FIELD OF THE INVENTION

The present Application relates to the field of sports training, and more specifically, to kinematic analysis of an athletic activity, such as a ball-striking event, captured on video or with any other sensor. More specifically, the Application relates to a system and method of kinematic analysis for one or more players, including detection of a ball-strike, performance of pose estimation on an image of a player, generation of a visual representation of a core system including one or more biomechanical execution parameters related to the ball strike, overlay of the representation on the pose points of the player, and imparting lessons regarding improvements in performance.

### BACKGROUND OF THE INVENTION

Many different techniques have been implemented in order to teach the proper mechanics of various athletic motions, in particular athletic motions involving striking a ball. Examples of sports that require such athletic motions include tennis (hitting a ball with a racket); table tennis (hitting a ball with a paddle); golf (hitting a ball with a club); baseball (hitting a ball with a bat); and cricket (hitting a ball with a bat). In the discussion that follows, tennis instruction is provided as an example.

Many instructors use a video analysis system to teach a student how to properly swing a tennis racket. Using a typical video analysis system, the student's stroke is captured by a video-recording device. The instructor replays the recorded video information to illustrate the student's stroke while providing feedback regarding the swing.

Although video analysis systems are widely used by tennis professionals, these systems have drawbacks. One drawback relates to having a professional subjectively analyze the video information. Not only is this analysis subjective and therefore open to interpretation and subject to inaccuracies, but also such analysis is exacerbated by the fact that many problems associated with a stroke are typically not captured by the video recording system.

In order to overcome the drawbacks associated with typical video analysis systems, instructors have adopted motion analysis systems as an aid to analysis and instruction. Current motion analysis systems require that the student/athlete wear sensor elements on their body and the sensor elements transmit positional data of isolated body parts, such as hands, hips, shoulders and head. The isolated points on the body are measured during a stroke in accordance with an absolute reference system, e.g., a Cartesian coordinate system wherein the center point is a fixed point in the room. By using motion analysis, exact measurements are provided from which an instructor can more accurately determine problems in a student's stroke.

Even though motion analysis provides accurate positional data of the student's stroke, it is not, in and of itself, particularly useful since it, too, gives no visual aid as to where the problems may really be. When used by itself, the motion analysis system is not an effective teaching tool since the instructor is only provided with numbers, and not a visualization of what the student is doing wrong.

In addition, the hardware requirements of such systems render them inaccessible to most students. Typically, these systems rely on a combination of: (1) IMU sensors and/or reflective markers placed on the player's body and ball-striking implement (e.g., racket, bat, club); and/or (2) fixed cameras placed around a playing field or court. These implements are expensive, time-consuming to set up, and of limited availability.

### SUMMARY OF THE INVENTION

It is an object of the present disclosure to provide a pictorial form of feedback regarding performance of a physical activity, such as a ball-strike, from which a player or coach may immediately understand the quality of the activity or ball-strike, without requiring reference to numerical data. It is a further object of the present disclosure to provide a system for identifying ball strikes and analyzing player performance based on analysis of a single video taken from a standard mobile device, without requiring any additional hardware.

The present disclosure describes a method for analyzing and providing pictorial feedback regarding biomechanics of ball-striking or physical events. The method begins with receipt of a video of a player taking a shot. The video may be taken from a single mobile device. Pose estimation is performed on the player, and the different pose points are converted to signals. On the basis of movement of the pose points, different shots are identified within the video. For each shot, a pictorial representation of the pose points before, during, and after the shot is generated.

In order to provide feedback regarding different aspects of kinematic performance, this pictorial representation is rotated or presented within different planes. In especially advantageous embodiments, the pose points are viewed on the X-Z plane. Geometric shapes or other signifiers may be overlaid onto the pose points in order to emphasize different aspects of the player's body orientation during the shot. Pictorial feedback may also be overlaid on a video of the player during the ball-striking event. Multiple pictorial representations may be selected, in order to highlight different aspects of kinematic performance. In particular, aspects of kinematic performance may be highlighted that bridge the intersection between physics, bio-mechanics, and sports technique.

The pictorial feedback provided herein enables intuitive communication of biomechanical principles to players, in a way that traditional coaching and even known video analysis systems are unable to provide.

According to a first aspect, a method of performing kinematic analysis of a ball-striking event is disclosed. The method includes: receiving a video of a player during a ball-striking event containing a plurality of ball strikes; performing pose estimation on images of the player in multiple frames within the video, and deriving pose points of the player in each frame; determining a series of frames within the video in which a ball-strike occurs, for each series of frames in which a ball-strike occurs, orienting a view of the pose points in order to enable visual assessment of one or more execution parameters associated with kinematic performance of the player during one or more ball strikes within the ball-striking event; and displaying a visual representation of the one or more execution parameters overlaid on the oriented view of the pose points during the ball-striking event.

In another implementation according to the first aspect, the method further includes displaying a visual representation of the one or more execution parameters overlaid on a visual display representing the player during the ball-striking event.

In another implementation according to the first aspect, the determining step comprises converting positions of pose points of the player into signals on an X-Y-Z plot, wherein X represents lateral movement, Y represents up-and-down movement, and Z represents front-and-back movement; determining frames of interest based on at least one of lateral movement of the pose points within the X-direction and front-and-back movement in the Z direction; and, within the frames of interest, determining existence of a shot based on up-and-down movement of the pose points.

In another implementation according to the first aspect, the method further includes classifying a ball strike as a type of shot based on a comparison of pose point signals of the player during the ball strike to pose point signals of the player or different players in previously-classified ball-striking events.

In another implementation according to the first aspect, the one or more ball strikes are selected from a group consisting of: a tennis shot; a table tennis shot; a golf shot; a baseball swing; or a cricket bat.

In another implementation according to the first aspect, the video is a video captured by a handheld mobile device.

In another implementation according to the first aspect, the method further includes generating a time series of pose points corresponding to different stages of a ball strike.

In another implementation according to the first aspect, the one or more visual representations include a C-factor, representing extent of rotation of the shoulder and hips relative to a frontal plane of the player, and a V-factor, representing a variation of angle of separation between a shoulder line and a hip line.

Optionally, the method further includes representing the C-factor as an inner curve nested within an outer curve, wherein the inner curve represents rotation of the hips, and the outer curve represents rotation of the shoulders.

Optionally, the method further includes representing the V-factor as a time series of an angle of separation between the shoulder line and the hip line.

In another representation according to the first aspect, the method further includes representing a kinematic chain transferred through the player's joints during the ball-striking event.

Optionally, the kinematic chain extends from the player's legs, to the player's trunk, to the player's shoulder, to the player's elbow, to the player's wrist.

Optionally, the method further includes representing the kinematic chain as fully interlinked when kinetic energy is properly transferred from the legs to the wrist, and representing a break in the kinematic chain when kinetic energy is not properly transferred from the legs to the wrist.

In another implementation according to the first aspect, the one or more execution parameters comprises stability of a foundation of the player prior to a ball strike, wherein the foundation is measured as a ratio between a distance between the player's feet and the distance between the player's shoulders as viewed in the x-z plane.

In another implementation according to the first aspect, the one or more execution parameters comprises a pressure index prior to a ball strike, wherein the pressure index is defined as a change in a center of gravity of the player over a course of a defined period prior to a ball strike.

Optionally, the method further includes displaying the pressure index as a time-lapse depiction of weight transfer overlaid on a visual display representing the player during the ball-striking event.

Optionally, the time-lapse depiction comprises a set of lines, wherein each line corresponds to a single time unit.

Optionally, the set of lines is weighted in a gradient fade, in which lines corresponding to earlier time units are sequentially blurred relative to lines corresponding to later time units.

Optionally, the set of lines is applied to only a portion of the player's body.

In another implementation according to the first aspect, the one or more execution parameters comprises a shift in foundation post-swing.

In another implementation according to the first aspect, the one or more execution parameters comprises a weight transfer of a torso and head of the player, and the method further comprises displaying a triangle overlaying an image of the torso the player during the ball strike, and changing the display of the triangle based on whether the torso of the player is predominantly behind, in front of, or parallel to an ankle line over a course of a shot.

In another implementation according to the first aspect, the one or more execution parameters comprise a hip-neck stretch, and the method further includes displaying the hip-neck stretch as a distance between a mid-hip point and a neck on pose points representing the player during the ball-striking event.

In another implementation according to the first aspect, the one or more execution parameters include a symmetry of the player's body during the swing, and the method further includes displaying a polygon drawn by the player's shoulder's elbows, and wrists over the pose points viewed in the transverse plane during the ball-striking event.

According to a second aspect, a method of performing kinematic analysis of a ball-striking event is disclosed. The method includes: receiving a video of a player during a ball-striking event containing a plurality of ball strikes; determining a series of frames within the video in which a ball-strike occurs; and determining a pivot frame of a ball strike within the video, in which the ball is contacted by a ball-striking implement, wherein the step of determining the pivot frame comprises: selecting, for each of the frames in the series, signals representing pose points of body parts of the player that form an X-factor, said X-factor defined as an angle formed between said player's shoulder line and said player's waist line; comparing the signals forming the X-factor of the ball-strike with signals forming X-factors calculated from previously recorded ball-strikes; on the basis of said comparison, determining a best match of the ball-strike to one of the previously recorded ball strikes; and determining the pivot frame based on a comparison to a pivot frame of the best matched ball strike.

In another implementation according to the second aspect, the step of determining the series of frames further comprises: performing pose estimation on images of the player in multiple frames within the video; converting positions of pose points of the player into signals on an X-Y-Z plot, wherein X represents lateral movement, Y represents up-and-down movement, and Z represents front-and-back movement; determining frames of interest based on at least one of lateral movement of the pose points within the X-direction and front-and-back movement in the Z direction; and determining existence of a shot based on up-and-down movement of the pose points.

In another implementation according to the second aspect, the step of determining a best match comprises performing a Euclidean distance analysis of the signals making the X-factor compared to signals making the X-factors of the previously recorded ball-strikes.

Optionally, the step of determining a best match further comprises performing a dynamic time warping (DTW) algorithm to the signals from the ball-strike and the signals of the previously recorded ball strikes.

In another implementation according to the second aspect, the method further includes classifying a ball-strike as a type of shot on a basis of a comparison of pose point signals of the player during the ball-strike to pose-point signals of players in previously classified ball-striking events.

In another implementation according to the second aspect, the method further includes comparing one or more execution parameters of a ball strike to the same one or more execution parameters of the best-matched previously recorded ball strike.

Optionally, the one or more execution parameters includes a weight transfer of the player during the ball strike.

Optionally, the one or more execution parameters includes a foundation posture and a landing posture of the player during the ball strike.

Optionally, the one or more execution parameters includes a location of a contact point of a ball-striking implement with the ball relative to the player's body.

Optionally, the method further includes comparing a consistency of the location of the contact point with other locations of contact points during the ball-striking event.

Optionally, the one or more execution parameters includes a swing line during the ball strike.

Optionally, the one or more execution parameters includes a leg stance during the ball strike.

In another implementation according to the second aspect, the one or more ball strikes are selected from the group consisting of: a tennis shot; a table tennis shot; a golf shot; a baseball swing; or a cricket bat.

In another implementation according to the second aspect, the video is a video captured by a handheld mobile device.

According to a third aspect, a method of providing feedback related to kinematic performance of a player during a ball-striking event is disclosed. The method includes: comparing one or more execution parameters, associated with the kinematic performance of a player during one or more ball strikes within the ball-striking event, with the same one or more execution parameters of a previously recorded ball strikes; and displaying one or more visual representations of the one or more execution parameters as an integrative feedback display overlaid on a visual display representing the player during the ball-striking event; wherein the one or more execution parameters comprises weight transfer of the player during a ball strike, and the visual representation is a time-lapse depiction of the weight transfer.

In another implementation according to the third aspect, the time-lapse depiction comprises a set of lines, wherein each line corresponds to a single time unit.

Optionally, the set of lines is weighted in a gradient fade, in which lines corresponding to earlier time units are sequentially blurred relative to lines corresponding to later time units.

Optionally, the set of lines is applied to only a portion of the player's body.

In another implementation according to the third aspect, the one or more execution parameters further includes a foundation posture and a landing posture of the player during a ball strike, and the visual representation includes at least one of (1) a geometric shape extending between the player's feet; and (2) an animation extending from the player's knees to below the player's feet.

In another implementation according to the third aspect, the one or more execution parameters further includes a contact point of the ball with the ball striking implement, and the visual representation includes one or more lines conveying the impact point of the ball with the ball striking implement.

In another implementation according to the third aspect, the method further includes displaying educative information with respect to improving the one or more execution parameters.

In another implementation according to the third aspect, the visual display representing the player is selected from a group consisting of: a photo, a video stream, an animated avatar of the player, or any combination thereof.

According to a fourth aspect, a system includes a non-transitory computer readable medium having stored thereon software that, when executed by a processor, cause the processor to perform the following steps: receiving a video of a player during a ball-striking event containing a plurality of ball strikes; performing pose estimation on images of the player in multiple frames within the video, and derive pose points of the player in each frame; determining a series of frames within the video in which a ball strike occurs; for each series of frames in which a ball strike occurs, orienting a view of the pose points in order to enable visual assessment of one or more execution parameters associated with kinematic performance of the player during one or more ball strikes within the ball striking event; and displaying a visual representation of the one or more execution parameters overlaid on the oriented view of the pose points during the ball-striking event.

Optionally, the system further includes a display device, adapted to receive instructions for generating the visual representation from said processor, and to display the visual representation to a user thereof.

Optionally, the computer-readable medium, processor, and the display device are integrated in a single device.

Optionally, the single device is a handheld smartphone or tablet.

### BRIEF DESCRIPTION OF THE DRAWINGS

FIG. 1 depicts steps of a method for performing kinematic analysis of a ball-striking event, according to embodiments of the present disclosure;

FIG. 2A illustrates pose estimation formed on a player during a ball-striking event, according to embodiments of the present disclosure;

FIG. 2B illustrates the use of pose estimation to determine points in a video sequence in which a shot is taken, according to embodiments of the present disclosure;

FIGS. 3A and 3B depict an X-factor formed between a waist line and a shoulder line during a ball-striking event, according to embodiments of the present disclosure;

FIGS. 3C-3E depict the variation between the waist line and the shoulder line using a C-factor and a V-factor representation, according to embodiments of the present disclosure;

FIG. 4 provides a schematic representation of the center of gravity, line of gravity, and base of support of a player, according to embodiments of the present disclosure;

FIG. 5A-51 illustrate pictorial representations of stability of foundation prior to a player's shot, according to embodiments of the present disclosure;

FIGS. 6A-6F illustrate pictorial representations of weight transfer and pressure index during lateral movement, according to embodiments of the present disclosure;

FIGS. 7A-7D illustrate pictorial representations of shift of weight post swing, according to embodiments of the present disclosure;

FIGS. 8A-8F illustrate pictorial representations of torso weight transfer during a swing, according to embodiments of the present disclosure;

FIGS. 9A-9B illustrate pictorial representations of hip-neck stretch, according to embodiments of the present disclosure;

FIGS. 10A-10F illustrate pictorial representations of symmetries in stance prior to a shot, according to embodiments of the present disclosure;

FIGS. 11A-11D illustrate pictorial representations of symmetries in stance during a shot, according to embodiments of the present disclosure;

FIGS. 12A-12D illustrate pictorial representations of detection of the point of maximum kinetic energy during a swing, according to embodiments of the present disclosure;

FIG. 13 illustrates an exemplary side-by-side display of pose points of a player during a shot and a video of the player during the shot, according to embodiments of the present disclosure;

FIGS. 14A-14C illustrate aspects of the kinetic chain and kinematic chain as applied to a tennis shot, according to embodiments of the present disclosure;

FIGS. 15A-15C depict additional forms of visual feedback that may be provided to the player, according to embodiments of the present disclosure;

FIGS. 16A-16D depict a series of pictorial representations of weight transfer, contact point, and foundation during performance of a backhand tennis shot, according to embodiments of the present disclosure; and

FIGS. 17A-17F depict a series of pictorial representations of weight transfer, contact point, and energy flow during performance of a forehand tennis shot, according to embodiments of the present disclosure.

### DETAILED DESCRIPTION OF THE INVENTION

The present Application relates to the field of sports training, and more specifically, to kinematic analysis of an athletic activity, such as a ball-striking event, captured on video or with any other sensor. More specifically, the Application relates to a system and method of kinematic analysis for one or more players, including detection of a ball-strike, performance of pose estimation on an image of a player, generation of a visual representation of a core system including one or more biomechanical execution parameters related to the ball strike, overlay of the representation on the pose points of the player, and imparting lessons regarding improvements in performance.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

As used in the present disclosure, the term “ball-strike” refers to an intentional contacting of a ball with a ball-striking implement, in the context of playing a sport or training for a sport. The sport may be any sport played with a ball and a bat, racket, club, or paddle, such as tennis, table tennis, golf, baseball, or cricket. The term “shot” is used interchangeably with the term “ball strike.” The term “ball-striking event” refers to a series of ball-strikes captured in the same video. As noted above, while the examples described herein relate to analysis of ball-strikes, the techniques described herein are applicable to any activity that requires biomechanical analysis.

The term “kinetic chain” refers to the transfer of energy through different portions of a player's body during execution of a shot. For example, in the case of tennis, the kinetic chain begins in the legs, and proceeds through the trunk and back, shoulder, elbow, and wrist. The term “kinematic chain” refers to a linking of joints through which force is transferred on the kinetic chain. For example, in the case of a tennis shot, force is generated using the person's weight by pushing against the earth (Ground Reaction Force) and transferred through the ankle joints, knee joints, hip joints, spine, shoulder joint, elbow joint, and wrist joint. The kinematic chain corresponds to movement, and thus is visible, and may be portrayed in a pictorial representation.

As used in the present disclosure, the term “execution parameter” refers to a measurable or coachable indication of the quality of performance. Each sport or activity has its own important execution parameters. In tennis, execution parameters may include weight transfer, foundation, landing, contact point, leg stance, and swing angle. In addition, every physical activity has basic related biomechanical parameters, including base of support, center of gravity, and balance.

The analyses described herein may be performed by a system having a processor and a non-transitory computer-readable medium having stored thereon software instructions, that when executed by the processor, causes the processor to perform various functions, as set forth herein. The system may be housed on a single computing device or may include multiple computing devices. The system may include a display device that is adapted to receive instructions for generating visual representations from the processor, and to display the visual representations to a user.

In exemplary embodiments, the system is housed on a single, handheld, mobile device such as a mobile telephone or a tablet computer. The mobile telephone or tablet may have an application (“app”) installed thereon that is configured to capture and/or receive videos of ball striking events or other physical activities, analyze the videos of the ball-striking events or physical activities, and display feedback and coaching instructions to the user via a screen of the mobile device.

Alternatively, the steps of capturing a video, performing the kinematic analysis, and displaying the feedback may be divided among more than one computing device. For example, the video may be captured by a first device, and sent via a network to a computer that performs the kinematic analysis. The computer prepares feedback and sends the feedback via the network to the mobile device, where the feedback is displayed.

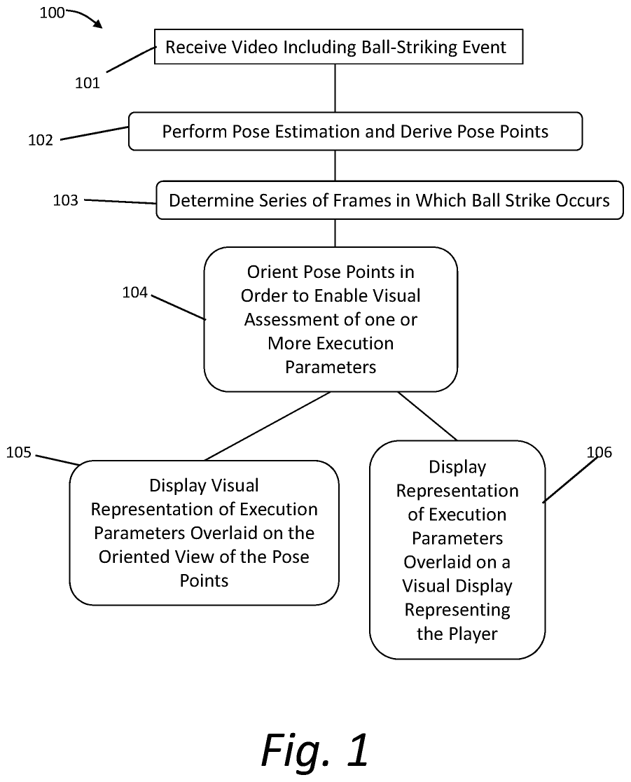

FIG. 1 depicts steps of a method 100 of performing kinematic analysis of a ball-strike, according to embodiments of the present disclosure.

At step 101, the system receives a video of a ball-striking event. The video may be captured by the same device that performs the subsequent analyses. The video may alternatively be sent to a different computing system for performance of the subsequent steps.

In preferred embodiments, the video is captured at a frame rate of at least 25 frames per second.

At step 102, the system performs pose estimation and derives pose points at each frame of the video. The system performs this pose estimation by using image processing technology to determine the presence of persons within the video frames, and determine the location of joints and limbs of the persons, including tracking the location of the joints and limbs from frame to frame, in a manner known to those of skill in the art.

At step 103, the system determines a series of frames in which a ball strike occurs. In the description that follows, determining the frames in which a ball strike occurs is explained with reference to a tennis shot. Similar principles may be used for determining the presence of a shot in other sports as well, including table tennis, golf, baseball, and cricket.

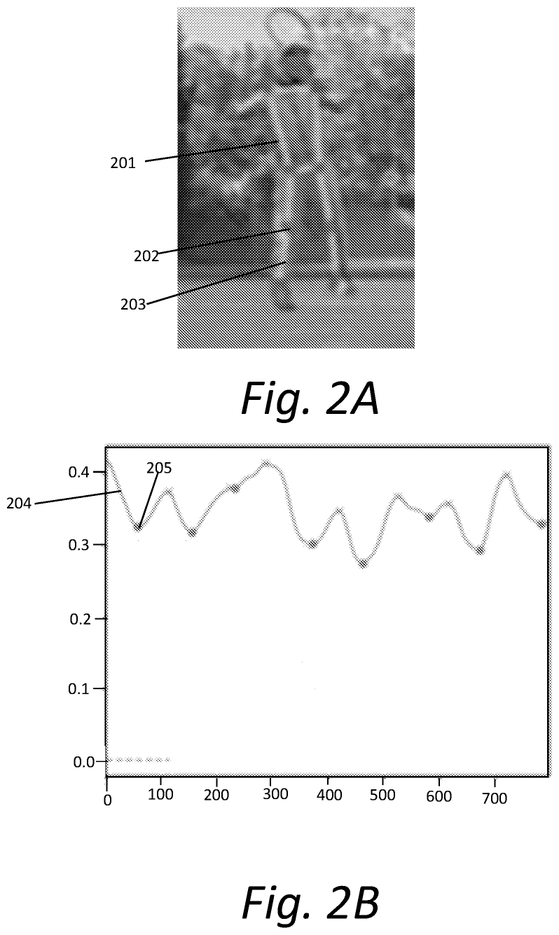

Referring to FIG. 2A, pose estimation analysis is performed on a player 201 playing tennis. The pose estimation abstracts from a video of the player a series of points 202, corresponding to joints, and a series of lines 203 connecting between the points, corresponding to limbs. In exemplary embodiments, 18 pose points are chosen. The number of points may be greater as well, for example, 25 points, or 32 points, or any number of pose points that is currently, or may eventually, be used.

The movement of a player on the tennis court may be imagined in three axes, with the X axis being the left-to-right direction (baseline to baseline), the Y-axis corresponding to the player's height, and the Z-axis being the front-to-back direction (back line to net, depth). The playing of tennis typically features lateral movement of the waist along the X axis between shots. During the shot, there is either lateral movement of the hand or a vertical burst of shoulder movement on the Y-axis.

Thus, in order to determine a series of frames in which a ball strike occurs, the system converts the pose positions into a series of signals on the X-Z axis, and maps a time series of the player's lateral movements on one or both of the X and Z axes. As shown in FIG. 2B, this time series may take the form of a graph that looks akin to a sine wave, with curve 204 representing the periods of movement, and inflection points 205 representing the periods of slowing or stopping to take a shot. Although, in the illustrated Figure, only a single line graph is shown, in practice a separate line graph may be generated for each data point 203 generated from the pose estimation.

In order to complete the analysis of whether a shot occurred, as opposed to random strolling on the court, or stopping to pick up a ball from the ground, the up-and-down movements of the torso arm segments and the hand movements are tracked on the Y-axis. Specifically, the maxima and minima of the “midWaist” are calculated, and a threshold is applied for maximum standard deviation of the hand movement. Alternatively, a more robust machine learning model is used to identify this pattern or a region of interest, using computer vision techniques or a hybrid model. The “hand movement” refers here to the pose points that include the hand and arm, such as “RightShoulder,” “RightElbow,” and “RightWrist.” When the mean of the hand movement on the Y-axis is below the waist, it is determined that the user was picking up a ball, as opposed to taking a shot. If the mean is above the waist, it is determined that the user was taking a shot. Optionally, in order for a shot to be registered, the standard deviation of the positioning of the hand relative to the mean may be required to be below a predetermined threshold.

This process may be repeated throughout the video. Following completion of the process, a series of frames of interest is identified, wherein each series of frames of interest contains therein a single shot. For each frame of interest, a series of signals is stored. The signals represent the three-dimensional locations of each of the pose points within the tennis court.

In advantageous embodiments, the sport that is being played is identified prior to the performance of the step of identifying frames of interest. Identification of the sport reduces the type of patterns of movements that are required to be analyzed. However, it is also possible to detect simultaneously both the sport and the presence of a shot.

As an alternative to the process defined above, instead of the system automatically determining the frames in which a shot occurs, the user may manually define the frames in which a shot or physical activity of interest occurs.

At step 104, the system orients pose points in order to enable visual assessment of one or more execution parameters. The pose points exist in three-dimensional space. Thus, it is possible to view the pose points from any vantage point and any plane. In particular, it is not necessary for the vantage point of the pose points to be the same as the vantage point of the camera that shot the video.

At step 105, the system displays a visual representation of execution parameters overlaid on the oriented view of the pose points. Specifically, the system displays various geometric shapes within views of the pose points from different vantage points. The geometric shapes correspond to the interrelationship between different pose points before, during, and after the execution of the shot. The geometric shapes provide intuitive feedback to the player regarding the user's positioning prior to, during, and after the shot. Examples of these geometric shapes are described herein.

At step 106, which is performed in addition to or instead of step 105, the system displays a representation of execution parameters overlaid on a visual display of the player. For example, during a video playback of the player taking a tennis shot, the system overlays on an image of the player graphics representing pose points, lines connecting the pose points, and geometric shapes that accentuate aspects of the player's performance.

Various examples of depiction of execution parameters are described below.

### Rotation of Shoulder Line Relative to Waist Line—X-factor, C Factor, and V Factor

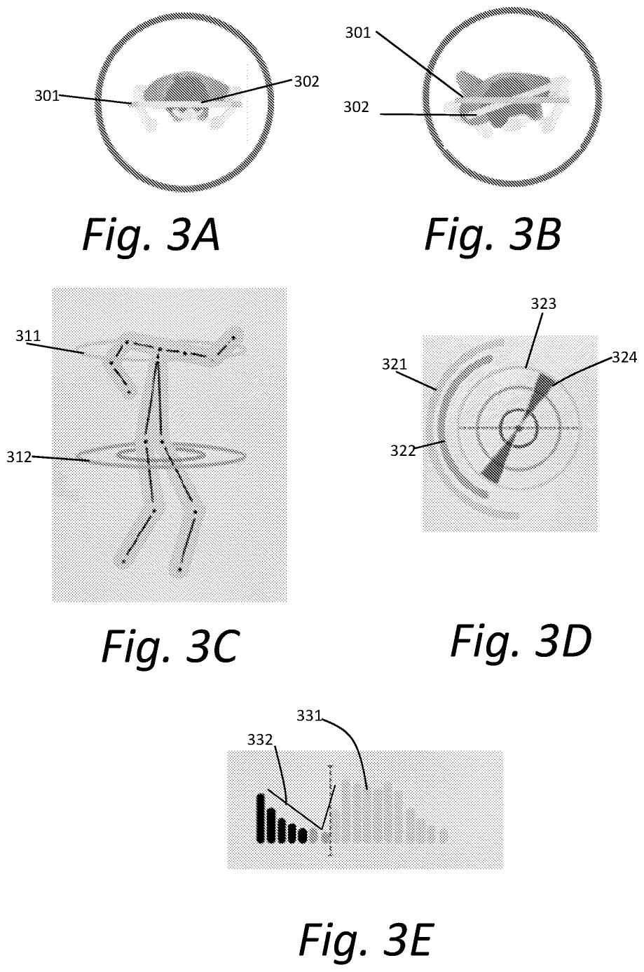

The term “X-Factor” refers to the angle formed between the shoulder line and the waist line during a shot. This angle is illustrated in FIGS. 3A and 3B.

FIG. 3A depicts an overhead view of a waist line 301 and shoulder line 302 of a player at rest. Specifically, the shoulder line and hip line are projected onto the transverse plane (the plane that is parallel to the ground). Because the player is not taking a shot, the two lines overlap. FIG. 3B depicts an overhead view of a player taking a shot. The shoulder line 302 is now angled relative to the waist line 301, such that the two lines form an X.

The X-factor has long been used in sports training as a metric indicating power transferred during the shot. In particular, sports literature speaks about maximum separation of the shoulder line and hip line for generating maximum force. However, it is difficult for a typical player to grasp how the X-factor relates to the quality of a shot.

The present disclosure presents a new graphic representation for the angle between the shoulder line and the waist line: the C-factor. The letter “C” refers both to the shapes represented on the graphic and to the words “Coil” and “Recoil.” Referring to FIG. 3C, during a shot, both the waist line and the shoulder line rotate relative to the Y-axis (vertical axis that passes through the middle of the shoulder line and middle of the waist line). In a properly executed shot, the rotation of the shoulder line 311 is around a wider angle than rotation of the waist line. An ideal swing has the shoulder lead first (for example, in a clockwise direction for a forehand in tennis on the loading of the shot (or “coil”). During the recoil action, the hip line leads (again, in a clockwise direction for a forehand in tennis).

As shown in FIG. 3D, the rotation of the shoulder line is illustrated as a large “C” 321. The upper apex of “C” 321 represents the maximal rotation of the shoulder line during the preparation to swing (“coil”). The lower apex of “C” 321 represents the maximal rotation of the shoulder line following the swing (“recoil”). Similarly, the rotation of the waist line is represented as small “C” 322, nested within the larger “C” 321, in which the upper apex of “C” 322 represents the maximal rotation of the waist line during the preparation to swing (“coil”), and the lower apex of “C” 322 represents the maximal rotation of the waist line during a follow-through of the swing (“recoil”). For ease of visual recognition, each of C's 321, 322 are oriented on a curve around circle 323. Circle 323 also has included thereon wedges 324, which represent the X-factor, i.e., the angle between the shoulder line and waist line during the shot.

FIG. 3E introduces another pictorial representation of the relationship between the shoulder line and the waist line-the V-factor. The term “V-factor” refers both to the shape of the pictorial representation and to the term “variation,” which refers to the variation of the angle of separation between the shoulder line and the hip line. Chart 331 indicates the variance between the shoulder line and the waist line throughout the process of a shot. In a properly executed shot, this variance, when plotted over time, looks like a “v,” indicated on the chart 331 with reference numeral 332—with the variance being greater before the shot and after the shot, and at its lowest point at the moment of the shot. Beginners and intermediate players, by contrast, do not always exhibit this “v” shape. The “v” factor may thus help beginner and intermediate players improve their game.

### Foundation

“Foundation” refers to the stability and posture of a player during a swing. Foundation may be broken down into two aspects: a stance phase, which is the posture of the player, and a foundation box, which refers to a box surrounding a player's feet, also called “BOS” or Base of Support.

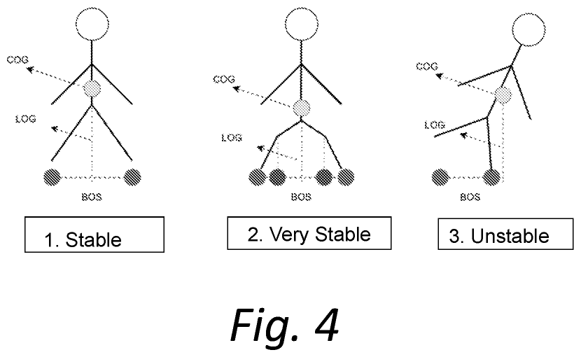

Regarding stance phase, typically, coaches instruct their students to “get wide and stay low.” The reason for getting wide and staying low is that this posture is the optimal posture for transferring energy from the legs to the wrist. The basis for this principle is illustrated in FIG. 4. In physics, gravity is a fundamental interaction which causes all things with mass or energy to be attracted toward one another. The center of gravity (“COG”) is the point at which the gravitational force acts. For purposes of calculating gravitational pull on the human body, it is assumed that the entire mass of the human body is concentrated on one place, which is the center of mass. The center of mass changes as the body changes from childhood to adulthood, and in an adult is anterior to the S2 sacral vertebra. For purposes of the present discussion, the center of gravity is assumed to be at the same location as the center of mass. The “Line of Gravity” (LOG) refers to the gravitational force vector on the body (i.e., the vector from the center of gravity to the ground). The “Base of Support” is the point where the human body touches the ground to maintain posture (typically, in tennis, the feet).

Stability refers to the resistance of the body to movement. Stated differently, stability refers to a measure of the equilibrium of a particular position. It is the nature of an object to return to equilibrium after being displaced. In the human body, factors affecting stability include the size of the base of support (BOS), the horizontal distance between the center of gravity and the edge of the base of support, the height of the center of gravity, the mass of the body, and the direction of the base of support in relation to the force. In particular, the height of the center of mass directly relates to stability, because lowering the center of mass causes a corresponding lowering of the torque caused by a lateral force.

Balance refers to the ability to maintain the line of gravity of a body within the base of support with minimal postural sway. When a body is balanced, opposing forces are equal, so that the opposing forces effectively cancel each other out. As a result, stability is maintained. The different relative locations of the center of gravity, line of gravity, and base of support determine the stability of a particular position.

At the left side of FIG. 4, the person is standing straight. The center of gravity is in the midsection, and the line of gravity is directed to a midpoint of the base of support (the feet). This position is stable. In the middle of FIG. 4, the person is flexing his knees, following the coaching advice to “get wide and stay low.” The center of gravity is lowered relative to the anatomical position. Lines of gravity are extended both from the midsection and from the flexed knees, and all of the lines of gravity fall within the base of support. As a result, this position is even more stable than that of the left side. On the right side of FIG. 4, the person is off balance. The center of gravity remains at the midsection, but is no longer oriented over the base of support. As a result, this body is not in balance, and will need to move in order to restore balance.

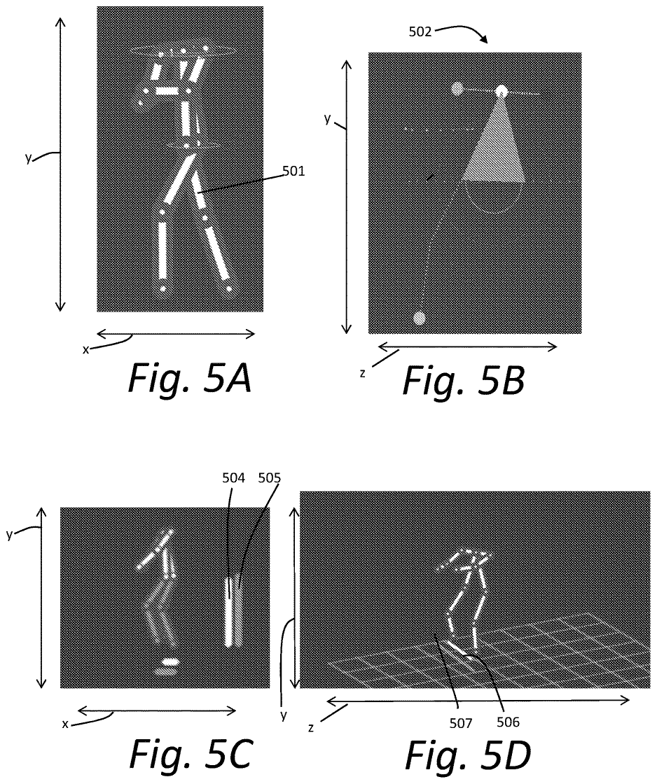

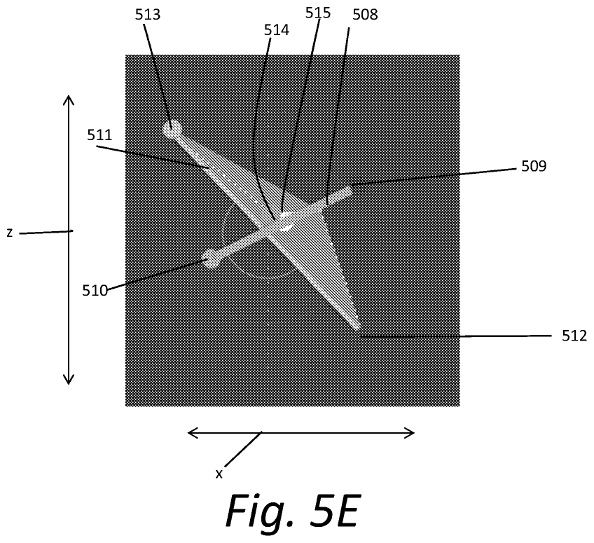

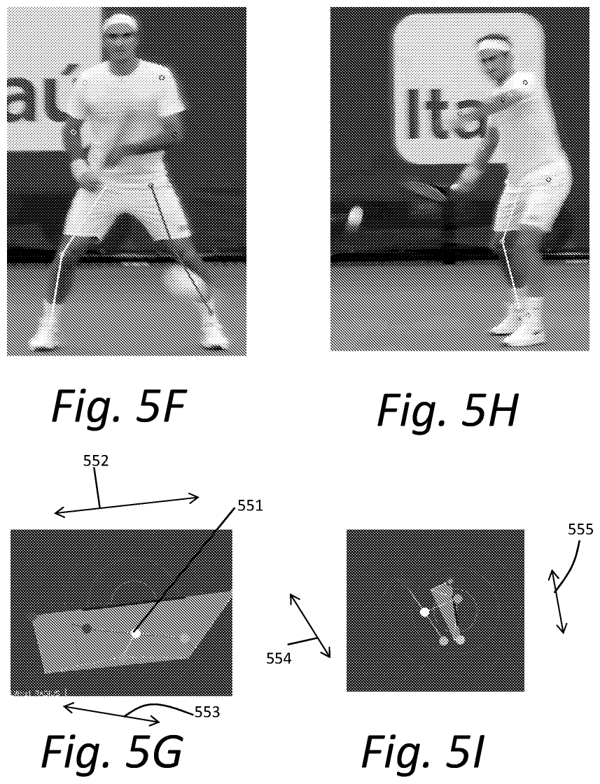

FIGS. 5A-5E illustrate different representations of pose points of the player that enables an intuitive understanding of stability and balance during the shot. Specifically, as discussed above, in order to obtain an intuitive understanding of the player's balance, it is necessary to consider the relationship between the center of gravity and the base of support. This, in turn, may be visualized by reference to the ratio between the distance between the shoulders and the distance between the feet. The length of the feet separation is around 1.3-1.7 times the shoulder length, when viewed in the x-z (overhead) plane. A larger ratio indicates a more stable position, and a smaller ratio indicates a less stable position. The notion of “stable position” varies from sport to sport. For example, this ratio is 1.3-1.7 in tennis, but it is 1.0 in golf.

FIG. 5A illustrates pose points of the player 501 taking the shot in the camera view of the x-y dimension, while FIG. 5B illustrates pose points 502 of the same player, with the view rotated to the y-z plane. In FIG. 5C, line 504 represents the actual feet separation in the x-y axis, and line 505 represents the ideal stable separation between the feet, as viewed in the x-y plane. This view in the x-y plane (shown in FIG. 5A and FIG. 5C) does not help in suggesting this ideal separation because, in that camera angle, it is hard to see whether the player is balanced. In FIG. 5D, the same distances are viewed from the y-z plane. As can be seen, line 506 representing the shoulder line ratio is significantly shorter than line 507 representing the line between the feet, indicating that the player is stable. It is required to rotate the pose points of the skeleton until this information can be shown to the user correctly.

FIG. 5E depicts the same analysis from the perspective of the x-z plane (i.e., an overhead view). The x-z plane provides the best view for analysis of stability and balance. This view is preferred because the shoulder and ankle movements in this plane may be viewed throughout the shot without being obscured by other body parts. It may be desirable to use other views in sports or activity where the shoulder line is constant in that view, such as yoga. In FIG. 5E, line 508 extends between left shoulder 509 and right shoulder 510. Line 511 extends between left foot 512 and right foot 513. Also visible are pose points for the center of gravity 514 and head 515. Thus, display of the view of FIG. 5E, along with data regarding the ratio of feet separation to shoulder separation, provides intuitive feedback to the player regarding foundation.

FIGS. 5F-51 provide further examples of the advantages of viewing the foundation in the X-Z plane. In FIG. 5F, a professional tennis player is depicted in the X-Y plane as preparing for a shot. This player has his feet spread wide apart, in a stable stance. FIG. 5G illustrates pose points 551 of the same player in the X-Z plane. Line 552 represents the distance between the feet, while line 553 represents the distance between the shoulders. FIG. 5H illustrates the same professional tennis player, in the X-Y plane, at the beginning of a shot. FIG. 51 illustrates the same player in the X-Z plane, with line 554 representing the distance between the feet, and line 555 representing the distance between the shoulders. Notably, while it is difficult to appreciate the distance between the feet in the view of FIG. 5H, it is quite easy to do so in the view of FIG. 51.

### Weight Transfer and Pressure Index

“Weight transfer” refers to the transfer of the center of gravity of player throughout the process of a shot. “Pressure Index” is a measure of the distance of the movement of the player during the shot, as indicated particularly through movement of the center of gravity in the lateral axis. A higher pressure index signifies that the player has moved a greater distance to reach the point where the later part of the swing is to be executed.

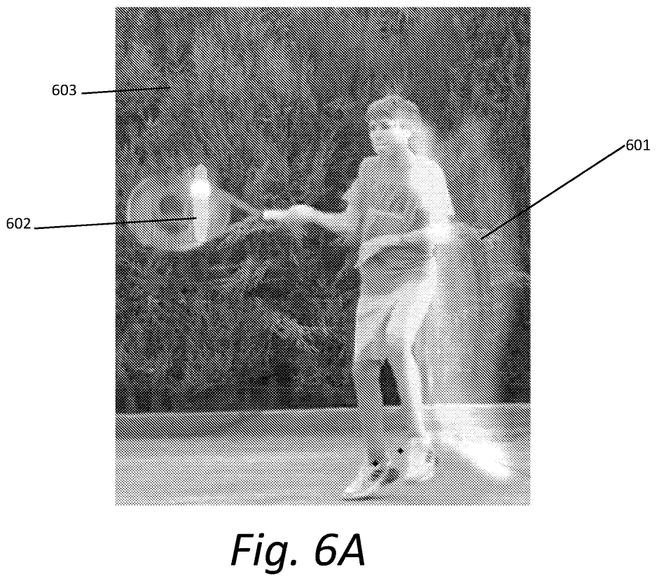

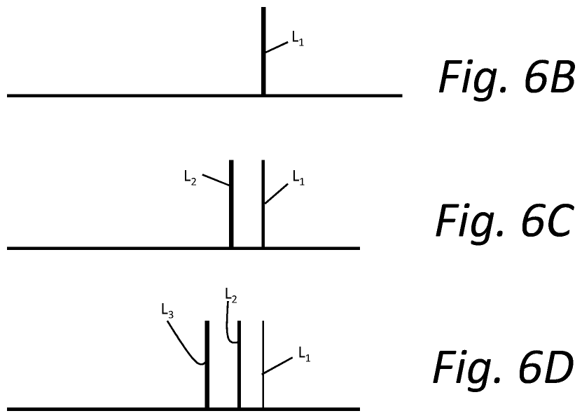

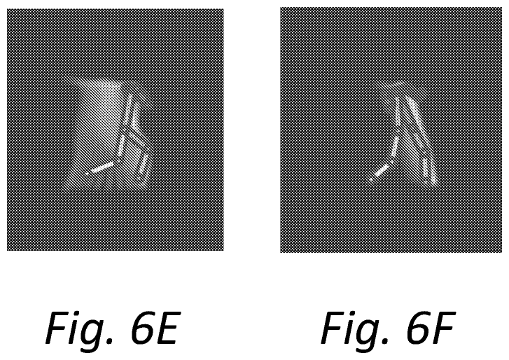

In FIG. 6A, the pressure index 601 of the player is illustrated, in a two-dimensional image, as a series of lines. Each line represents a center of gravity of the player beginning from the start of the shot, and continuing to through the end of the shot. Also, in this view, the location of the contact point of the ball and the racket is highlighted. In the illustrated example, the contact point is indicated with an ellipse 602. The background portions 604 of the video are displayed in black and white or otherwise distinguished when the visual representations are overlaid on the video, so as to maximize the viewer's focus on the visual representations. These representations may differ in a 3D environment.

The visual indicator 601 may be generated in various ways. In one approach, the visual indicator 601 for the weight transfer is based on the actual location of the center of gravity of the player. This center of gravity may be calculated from the pose points derived from each frame of the video, as discussed above. Each vertical striation in visual indicator 601 corresponds to the center of gravity during a single time unit. In order to make the display of the vertical line more prominent, the striation may be displaced to one side of the player's body, for example, the side opposite the direction of movement.

In addition, in order to indicate direction of the weight shift over time, the set of lines is weighted in a gradient fade. In operation of the gradient fade, striations corresponding to earlier time units are sequentially lightened or blurred relative to lines corresponding to later time units. This is illustrated at FIGS. 6B-6D. In FIG. 6B, a first frame shows line l₁ at t₁ which is displayed at a brightness level b₁₀₀. The brightness level is represented by the thickness of the line. In FIG. 6C, at t₂, the center of gravity is now at line l₂, to the left of l₁. The display shows both l₁ and l₂; however, l₂ is displayed at brightness b₁₀₀, whereas l₁ is displayed at brightness b₉₉. In FIG. 6D, after an additional frame, there are three lines l₃, l₂, and l₁, with respective brightness of b₁₀₀, b₉₉, and b₉₈. Consequently, the newer lines are always presented more strongly than the older lines.

To be clear, the gradient fade of the pressure index described here is fundamentally different from gradient fades used in computer vision, which depict later movements more strongly than earlier movements. In such cases, the gradient fade is applied to the entire image. Here, by contrast, the gradient fade is a visual representation of data derived from the pose points. The gradient fade is selectively applied onto only a portion of the body of the player, in order to create an aesthetically compelling effect.

FIG. 6E shows a player with a high pressure index. The player has moved a relatively large distance in the measured time period (for example, 1 second) before the shot. FIG. 6F shows a player with a lower pressure index. The player has moved a relatively short distance in the measured time before the shot. The pressure index may be presented as arbitrary numbers on a scale, e.g., between 1 and 10.

### Shift Post Swing

Another aspect of weight transfer relates to the shift in weight post-swing, in preparation for receiving the next shot. The post-swing shift may be “positive,” restoring the player to a balanced position, or a “negative” post-swing shift, as the context of the situation demands.

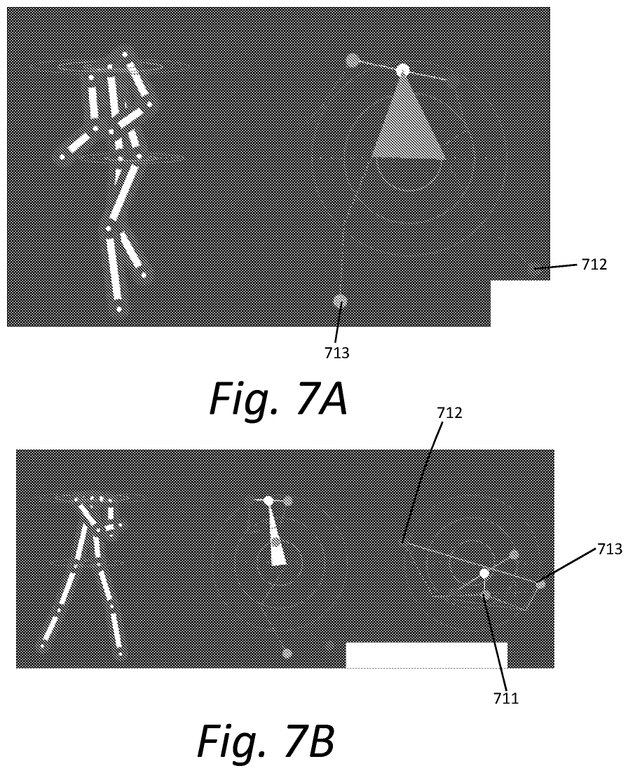

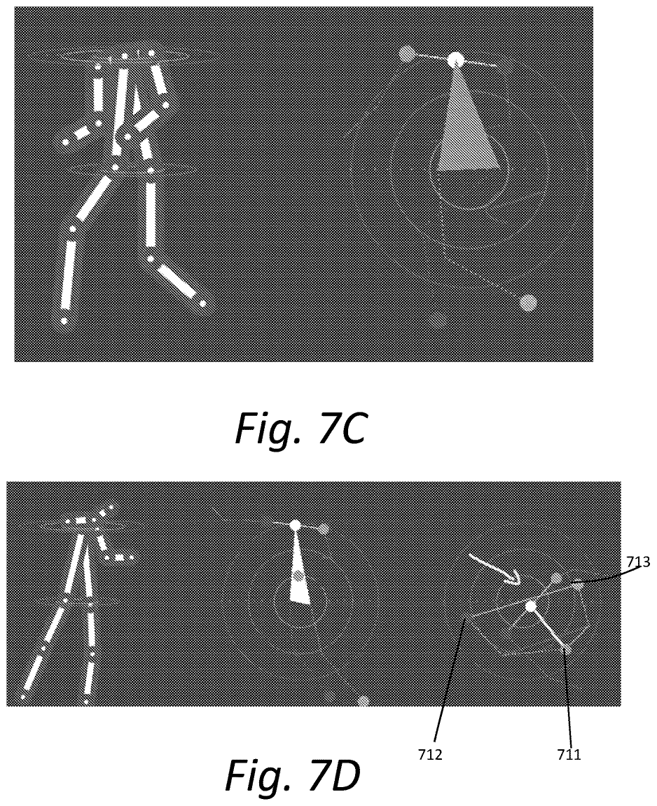

FIGS. 7A-7B illustrate the post-swing shift for a positive shift. The left side of FIG. 7A shows the pose points during the shot as viewed in the x-y plane, while the right side shows the same pose points in the x-z plane. FIG. 7B shows views of the pose points following the shot, in the y-z plane (left view and middle view) and the x-z plane (right view). As can be seen, especially from the right view of FIG. 7B, the resulting position of the player is balanced, with the center of gravity 711 relatively close to the line between feet 712 and 713, and the line between the feet significantly wider than the line between the shoulders.

FIGS. 7C-7D illustrate the post-swing shift for a negative shift. As illustrated with the arrow in FIG. 7C, the player takes the shot in an unnatural stance, with the legs crossed, and the right leg significantly behind the left leg. Since the stance of the player was problematic, the shift post-swing to correct is also problematic, as illustrated in FIG. 7D. As seen at the right of FIG. 7D, the center of gravity 711 is very far displaced from the line between feet 712 and 713.

### Torso Weight Transfer

Still another metric that may be displayed pictorially is weight transfer of the torso. ⅔ of the weight of the human body is in the torso and head. During a tennis shot, the torso is an important portion of the kinematic chain for transferring kinetic energy from the legs to the arms.

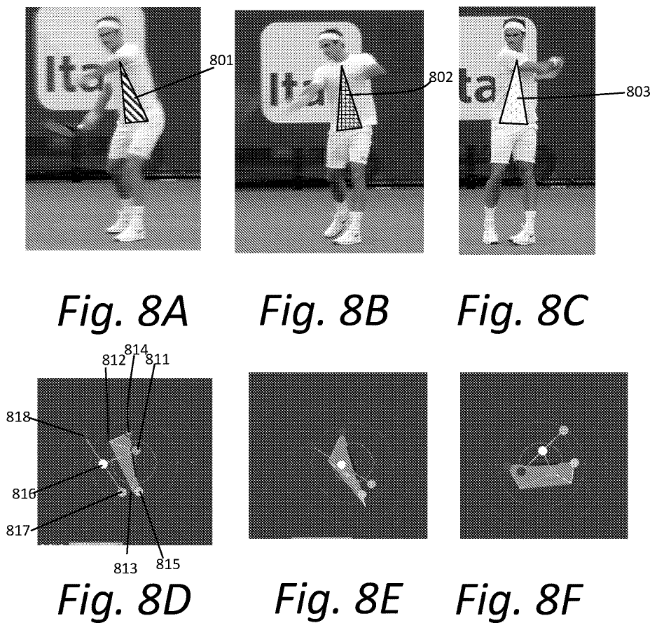

FIGS. 8A-8F illustrate a pictorial representation of the weight of the torso relative to the base of support. In FIGS. 8A-8C, a professional tennis player is viewed in the X-Y plane (camera plane). In FIGS. 8D-8F, the pose points of the same player in the same positions are viewed in the X-Z plane. In the view of FIG. 8D, the weight of the torso is tilted forward, toward the knee line. The center of gravity 811 is approximately between feet 814 and 815, while the head 816 and shoulders 817, 818 are in front of knees 812, 813. In FIG. 8E, the torso is evenly balanced over the feet and knees. In FIG. 8F, the weight of the torso is tilted backwards, behind the knees, as the head and shoulders are behind the line of the knees. The status of the tilt of the torso weight may be aggregated over the course of a shot or a time period. If, in the majority of instances, the torso tilts ahead of the knee line, a triangle in a first style 801 may be overlaid on a video of the player. If the torso is, on average, balanced above the knee line, a triangle in a second style 802 may be displayed, and if the torso is, on average, tilted behind the knee line, a triangle in a third style 803 may be displayed.

### Hip-Neck Stretch

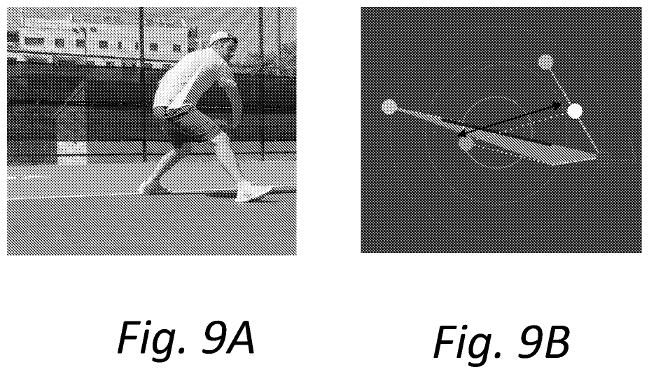

Referring to FIGS. 9A and 9B, there are times in which a player tilts forward or backward in such an extreme manner that it becomes counterproductive. This is because the body spends much of the energy generated from the lower extremities to regain balance and stability, rather than delivering power to the ball. This power needs to be compensated by the other parts of the kinematic chain, resulting in lower performance or injury. An example of an over-extended player is illustrated in FIG. 9A. In FIG. 9B, the pose points of that player are illustrated in the x-z plane. The unusually long extension (in the x-z plane) between the midhip and the neck is indicated with the arrow. This phenomenon may also be expressed numerically, as a ratio between the midhip-neck extension and the shoulder length.

### Body Symmetry

Generally, symmetry translates to conservation of energy, as proved by Noether's theorem. When an object is symmetrical, the center of gravity is assumed to be in the middle. For asymmetric bodies, the center of gravity is on the heavier side of the object. Because ⅔ of the body weight is in the torso and upper body, when the torso and upper body are not balanced over the angles, a “wedge effect” is generated. The wedge effect refers to the triangle generated between the base of support (the feet) and the center of gravity. The asymmetry generated in this wedge position in turn induces movement. However, it is difficult to visualize this wedge from a video shot in the X-Y plane.

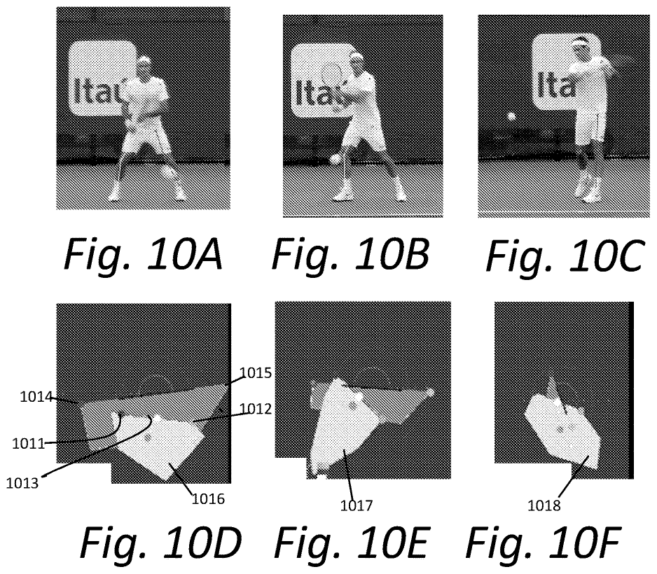

FIGS. 10A-10C illustrate a professional tennis player during the process of a shot, with an overlay of pose points of the weight-bearing limb or limbs. However, absent the overlay of the pose points, the balance of weight is not immediately apparent. FIGS. 10D-10F illustrate the pose points of the tennis player in FIGS. 10A-10C, projected into the X-Z plane. In FIG. 10A and FIG. 10D, the player is balanced evenly, with the weight (the line 1013 between the shoulders 1011, 1012) centered between the feet 1014, 1015 in the x-dimension. A wedge 1016 is drawn, representing the vectors of the weight distribution of the upper body. All vertices of the wedge are centered between the feet 1014, 1015 in the x axis. In FIGS. 10B and 10E, the weight of the player is shifted to the right leg. This is shown in FIG. 10E, with the shoulder line oriented mostly over the right side of the leg line from the perspective of the player, which is the left side of the leg line as viewed in the Figure. Correspondingly, a wedge 1017 is shown pointing from the shoulder line to the right. In FIGS. 10C and 10F, the weight of the player is on the left foot. The wedge 1018 extending from the shoulder line points to the player's left side.

Of course, during the course of play, the torso weight shifts between the right, left, and center. In addition to the instantaneous view of the torso weight vector though the wedge, it is possible to display cumulative values for the user, indicating the percentage of time that the user's torso weight vector was centered, directed to the left, or directed to the right.

### Symmetries in Swing

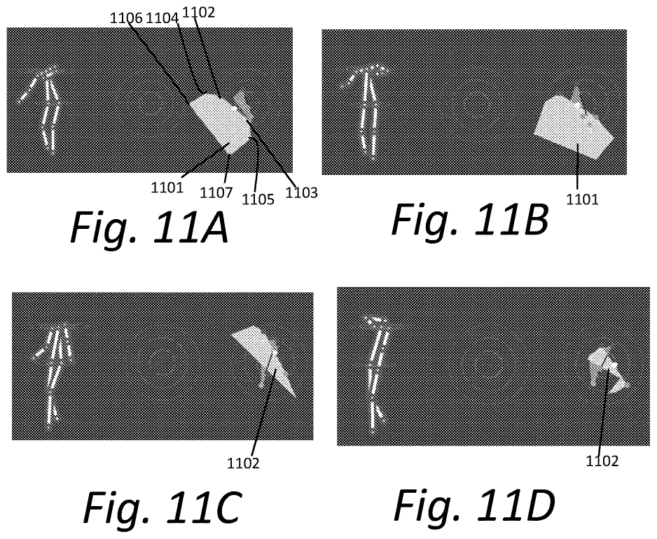

Another symmetry metric, based on Noether's theorem, that is possible to display intuitively using pose points and plane rotation is symmetry in the swing. A professional level swing is generally symmetrical and maintains its symmetry throughout the swing. This is illustrated in FIG. 11A and FIG. 11B. FIG. 11A shows, on the left side, pose points of a player during the beginning of a swing as viewed in the X-Y plane. On the right side, a projection of the pose points is shown in the X-Z plane. A polygon 1101 is drawn between the player's shoulders 1102, 1103, elbows 1104, 1105, and wrists 1106, 1107. This polygon is generally symmetrical. FIG. 11B shows pose points of the same player following the swing. Again, the polygon 1101 defined by the shoulders, elbows, and wrists is generally symmetrical.

FIGS. 11C and 11D illustrate the same views of pose points from before and after a swing, by an amateur player. The polygon 1102 defined by the shoulders, elbows and wrists is not symmetrical, neither before nor after the swing. As a result, the player is not effectively conserving energy generated from the lower links in the kinematic chain, and loses energy that could have been transferred to the shot.

### Contact Point

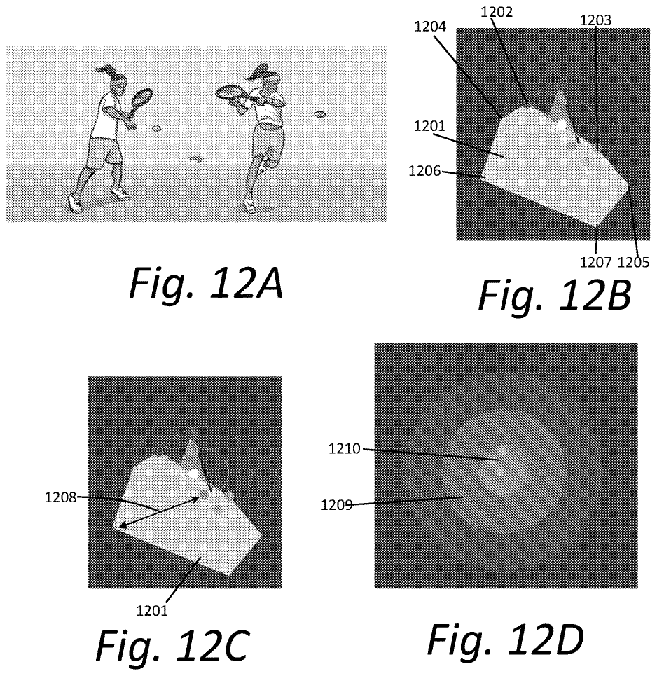

Contact point refers to the frame capturing the point in which the racket contacts the ball. More specifically, the metric of contact point defines the estimated position of the player in which kinetic energy is at its greatest. FIG. 12A depicts stages of a player's shot, from before the shot to after the shot. Both positions in FIG. 12A have high potential energy; by contrast, during the frame of the shot, the kinetic energy is at its highest.

Generally, at the contact point, the wrist is furthest extended from the shoulder line. Players aim to achieve consistency in arm extension during a series of shots.

The pose points may be used to calculate the contact point. Specifically, the frames identified as including the swing (as discussed in connection with FIGS. 2A and 2B), may be calculated based on maxima and minima of the wrist in the X and Z directions. At the frame that is determined to be the contact point, a polygon 1201 may be constructed around the shoulders 1202, 1203, the elbows 1204, 1205, and the wrists 1206, 1207. This polygon is illustrated in FIG. 12B. In FIG. 12C, arrow 1208 is drawn within the polygon, indicating the distance between the wrist and the shoulder line at the contact point. FIG. 12D is a scatter plot 1209 showing the extension of the wrist as a ratio of the distance between the shoulders. Each circle 1210 conveys the player's arm extension at the contact point for a different shot.

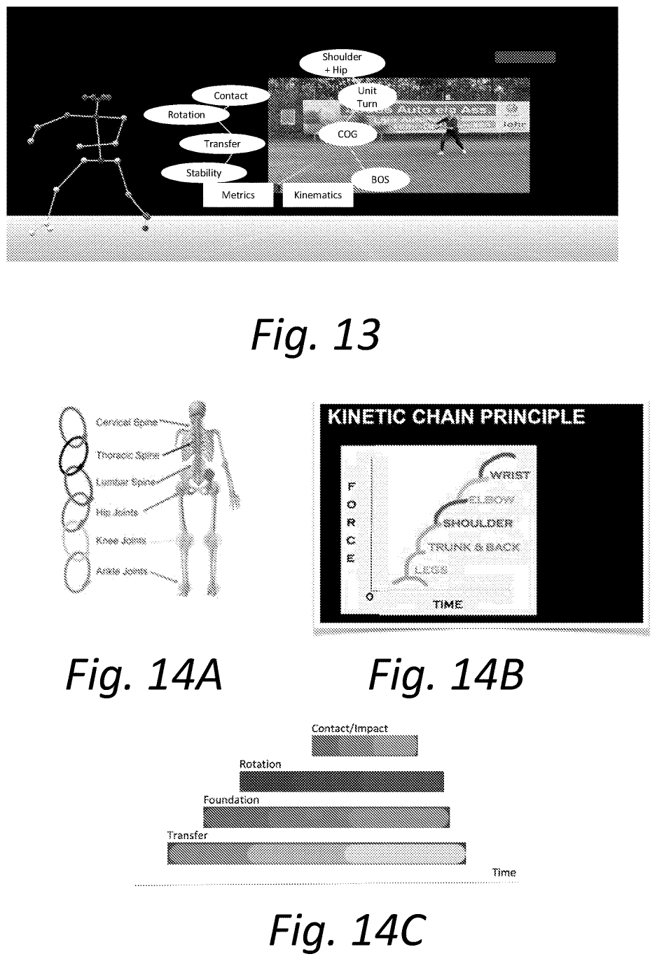

FIG. 13 illustrates an exemplary graphic user interface for viewing a graphical display of factors affecting performance of a player during a ball-striking event, according to embodiments of the present disclosure. The graphic user interface is a Game Engine, which is used to render 3D aspects of the pose estimation. The Game Engine is able to display metrics in all three planes-transverse, frontal, or sagittal.

At the center of the view of FIG. 13 is a playback of a video of a ball-striking event. On the left side of the screen, an avatar of the player is shown in pose points. Two menus are presented for the user, one focusing on kinematics (e.g., base of support, center of gravity, unit turn (rotation of shoulder line around ankle line)) and the other focused on metrics (e.g., stability, weight transfer, rotation, and contact point). The menus are presented in a “chain” in order to help the user understand how certain kinematic features depend on others, in a “building block” manner. Selecting an item from one of the menus enables the user to see one of the above-described feedback types overlaid on the screen (for example, in the location where the avatar is currently positioned, or in the opposite margin). The user interface may present both visual feedback (e.g., the C-factor, the V-factor, or the views of the pose points) and quantified feedback (e.g., ratios or scores), per the examples set forth above.

The menu or presentation may also indicate the link in the kinematic chain that breaches any predefined constraints, whether based on bio-mechanics or principles of sports. Additionally, the user may define specific combinations of feedback, or choose more than one fundamental metric to analyze per event. Furthermore, the system may display a comparison of a player's performance to those of ideal avatars or to an avatar representing the same player over time.

### Illustrations of Basic Principles

FIGS. 14A-14C illustrate principles of the kinetic and kinematic chains described above. FIG. 14A depicts the kinematic chain undertaken during a tennis shot. The body may be modeled as a series of pin-connected joints whose movements are coordinated and whose sequenced movement acts as the medium to transfer energy from the feet to the wrist. In the illustrated embodiment, the kinematic chain is depicted as fully interlinked, with each of the links present. When, during a shot, the energy is not properly transferred from the legs to the wrist, the kinematic chain may be represented with a break, indicating the link at which energy was not properly transferred.

FIG. 14B depicts the kinetic chain during a tennis shot. The kinetic chain represents how energy is transferred from the feet to the wrist via a well-coordinated sequence of body segments. Over the course of a shot, the player generates a force at the feet, transfers it to the thigh and core, and applies it at the wrist. FIG. 14C illustrates a time chart plotting, over time, the different aspects of kinematic performance as they are relevant during a shot. Weight transfer begins at the beginning of the shot process and extends through the end; during the course of the shot, the foundation, rotation, and contact or impact point become relevant.

### Comparison to Shots in Shot Library

In the previously described embodiments, the performance of the shot is evaluated without reference to other shots. In alternative embodiments, it is possible to compare shots to shots in a shot library and to issue a performance score based on the quality of the shot in relation to the closest-matched shot in the library. One way to compare shots to shots in the library is to use the X-factor as a basis for comparison. As described above, every player, during the course of a shot, generates an X shape through the intersection of the shoulder line and waist line, as viewed in the x-z plane. Signals making up the X-factor are compared to signals making the X-factors of the previously recorded ball strikes, for the duration of the frames of interest. Discrete Cosine Transform (DCT) may be used to match the signals based on the X factor on 2D. Specifically, DCT may be used to provide a transformation from the time domain to a set of COSINE transforms. The initial few coefficients serve as good candidates to understand the shape of signals of the body part movements. This is used to compare between the library of shots and the new shots.

One advantage of using the X-factor as a basis for comparison is that the computation necessary for comparing all the pose points of the player would be unduly cumbersome. A typical pose analysis considers 18, 25 or even more unique pose points. Comparing each of the pose points over the course of multiple frames, for each shot in a library, would result in a highly intensive computation, which could take many minutes. The X-factor is formed from fewer pose points. Specifically, while there may be a minimum of 18 pose points in general, the X-factor is made up of only 8 pose points. The shape of the X-factor is sufficiently different for each shot, such that these X-factor points themselves supply a sufficient basis for differentiation between different shots. Unexpectedly, experiments showed that there was no difference in accuracy between shot comparison on the basis of the X-factor and shot comparison on the basis of all of the pose points.

The system may then determine the best match of the ball strike to the previously recorded ball strike. The best match is determined through a comparison algorithm, such as a Euclidean distance algorithm. The Euclidean distance algorithm is used to measure the distances to the library shots in vector space. On the basis of the comparison algorithm, a percentage of matching is calculated for each shot. The shot with the lowest cumulative difference from the ball strike is determined to be the best match.

Once the closest match is identified, the pivot frame is also identified. The pivot frame refers to the frame in which the ball is struck. Typically, the library shots already have the pivot frame identified. As a result, the pivot frame may be identified through analysis of the time series pattern of the ball strike, in comparison with the time series pattern of the library shot.

Following identification of the pivot frame, other frames relevant to analysis of the shot may be identified. A set number of frames prior to the pivot frame may be identified as the frames relevant to weight transfer. In addition a “foundation” frame may be defined as a frame that is a set number of frames (e.g., two) before the pivot frame, and a “landing” frame may be defined as a frame that is a set number of frames (e.g., two) after the pivot frame.

### Shot Classification

In addition to identifying the best matched shot, the system may also classify the shot. The classifications of the shots are dependent on each sport. In tennis, the basic shots are forehand, backhand, and serve. There are more specific classifications based on playing style and percentage of movement within the court, such as aggressive baseliner (larger movement) or counterpuncher (more limited movement). There are also classification categories based on swing lines.

The core algorithm to classify the shots may involve a LSTM convolutional network and a sparse representation of the time series. Techniques such as FFT (fast Fourier transform), SVD (singular value decomposition), DCT (discrete cosine transform), and DTW (dynamic time warping) are used in tandem with the convolutional network to match the pose related to the frames of interest with a known classification category. In addition, as a shot library grows, it is efficient to use just a few shots to classify a category of shots, and then use matching techniques to narrow down to the finest match.

The following is a basic description of the training of an artificial intelligence network used to classify the shots. The description herein is one exemplary embodiment, and variations in the classifications and training may be implemented, without departing from the scope of the present disclosure.

Videos selected for the library are first profiled as adults or kids. Within the adults category, videos are classified as pro, semipro, and amateur, and within the kids category, videos are classified as advanced or amateur. Good shots are selected from these videos.

For each shot in the library, key frames are annotated. The key frames are start of shot, contact point (pivot frame), and end of shot. Pose points of all the frames between the “start of shot” frame and the “end of shot” frame are stored. Each shot is classified as backhand, forehand, serve, or volley. Adjectives such as “aggressive” or “counterpuncher” are also applied. Thus, in sum, the previously recorded ball strikes are classified on the basis of the following parameters: skill level, type of ball strike, and degree of power applied.

Once the library has been assembled and annotated, the artificial intelligence neural network is trained to classify new shots. When the training set is small, all of it is used, and an efficient pattern search is used with methods like the X-factor.

### Visual Feedback Including Video Overlays, Quality Scores, and Educative Information

As discussed above, in the previously described embodiments, the feedback is provided in the form of either pose points of the player as viewed through a particular plane, placement of particular geometric figures on the pose points, and/or overlay of pose points onto a video of the player. In alternative embodiments, the feedback is provided by overlaying various visual indicators onto the video of the players. These visual indicators may call attention to various execution parameters, such as foundation, landing, weight transfer, and contact point. In addition, the feedback may include quality scores or a quality scale regarding performance of the execution parameters. Finally, the system may display educative information that instructs more specifically about performance of the shot and on ways to improve the execution parameters. The system may also provide the user the ability to selectively choose the execution parameters for further analysis.

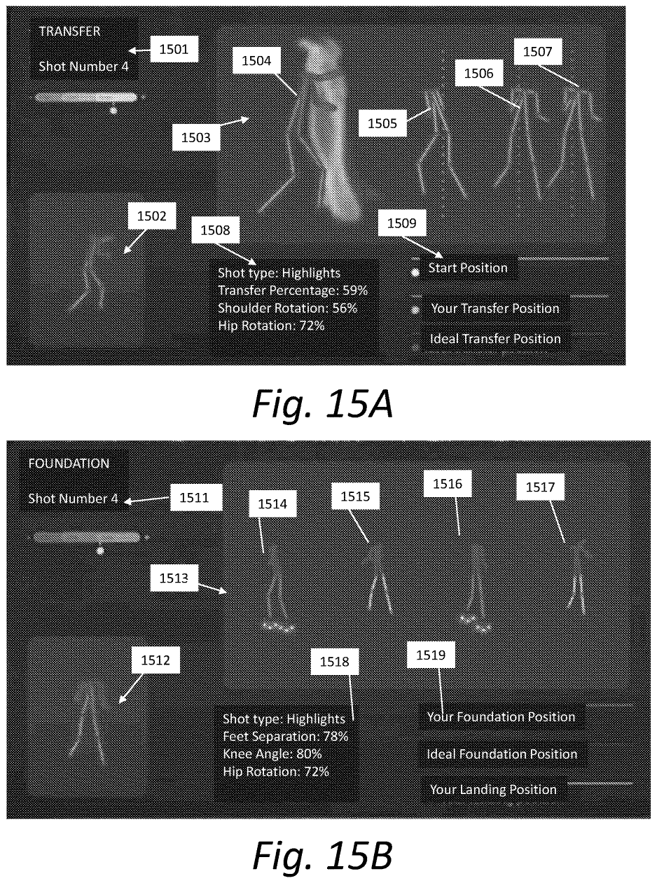

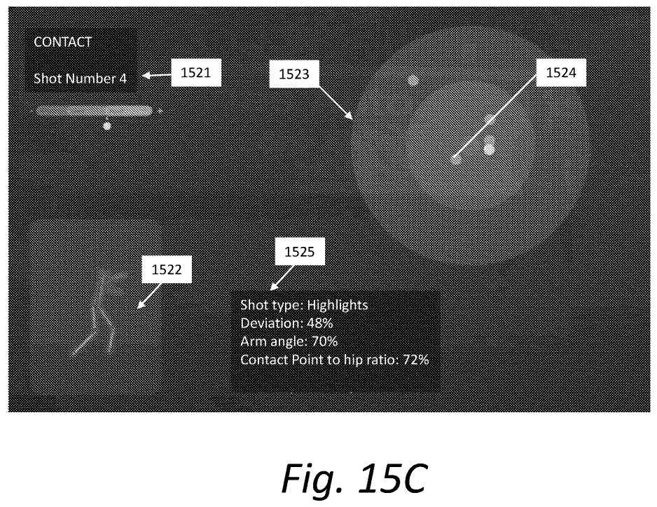

FIG. 15A is a graphic user interface that includes detailed feedback about weight transfer. The interface includes identification section 1501, which indicates which shot number is being analyzed within a ball striking event, which metric is being analyzed (in this case, weight transfer), and the overall quality of the weight transfer as measured on a sliding scale. Section 1502 includes an animation of the pose points of the player simulating the ball strike from beginning to end, which may repeat continuously during view of this graphic user interface. Section 1503 includes different views of the weight transfer as represented on figures generated by pose points, in which: FIG. 1504 shows the weight transfer striations progressing throughout the shot; FIG. 1505 shows the start position; FIG. 1506 shows the transfer position; and FIG. 1507 shows the ideal transfer position. Section 1508 includes statistics relevant to the execution of the weight transfer, including transfer percentage, shoulder rotation, and hip rotation, while section 1509 is a legend for section 1503.

FIG. 15B is a graphic user interface including detailed feedback about foundation and landing. Identification section 1511 indicates, similar to section 1501, the shot number in the sequence, the execution parameter that is being displayed (“foundation”), the shot number within the ball striking event, and the overall score for foundation and landing on a sliding scale. Section 1512 includes an animation of the pose points of the player simulating the ball strike from beginning to end, which may repeat continuously during view of this graphic user interface. Section 1513 includes different figures representing, respectively, the player's foundation position 1514, the ideal foundation position 1515, the player's landing position 1516, and the ideal landing position 1517. Metrics section 1518 includes educative information, such as specific scores for metrics that are related to foundation and landing, such as feet separation, knee angle, and hip rotation, again as compared to ideal positions. Additional feedback or educative information may be shown in separate screens. Section 1519 is a legend for section 1513.

FIG. 15C depicts a graphic user interface with regard to contact point. Identification section 1521 and animation section 1522 are similar to those described above in connection with FIGS. 15A and 15B. For contact point, it is considered important not only to match a contact point with an ideal contact point, but also to be consistent with contact points of previous shots. Accordingly, contact points 1524 from all of the shots in a ball-striking event are overlaid on target 1523. Similarly, the score that is provided for contact point is based on a combination. 80% of the score represents the comparison to the best-matched shot in terms of distance of the wrist to the waist. 20% of the score is derived from consistency to other contact points of previous shots by the same user. Metrics section 1525 includes scores for metrics relevant to contact point, such as deviation, elbow angle, and contact point to hip ratio.

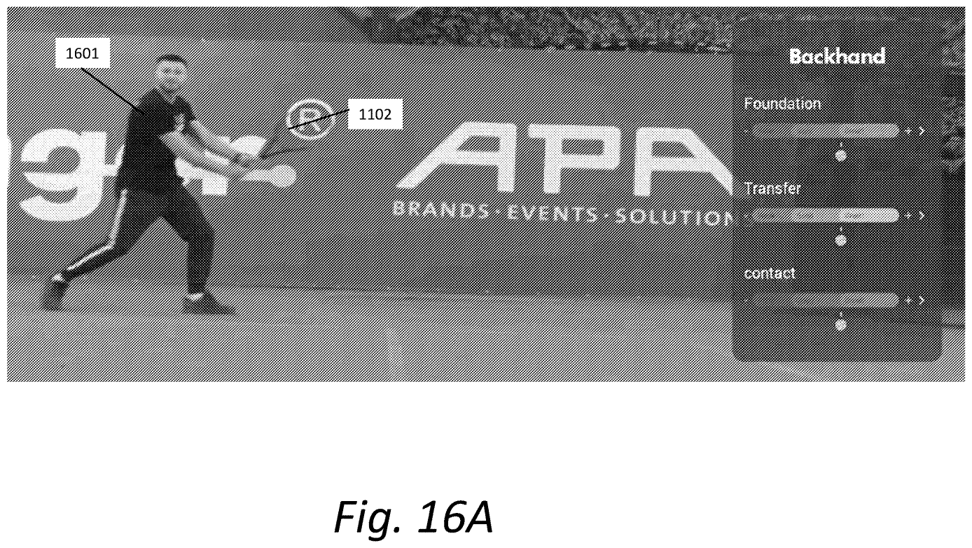

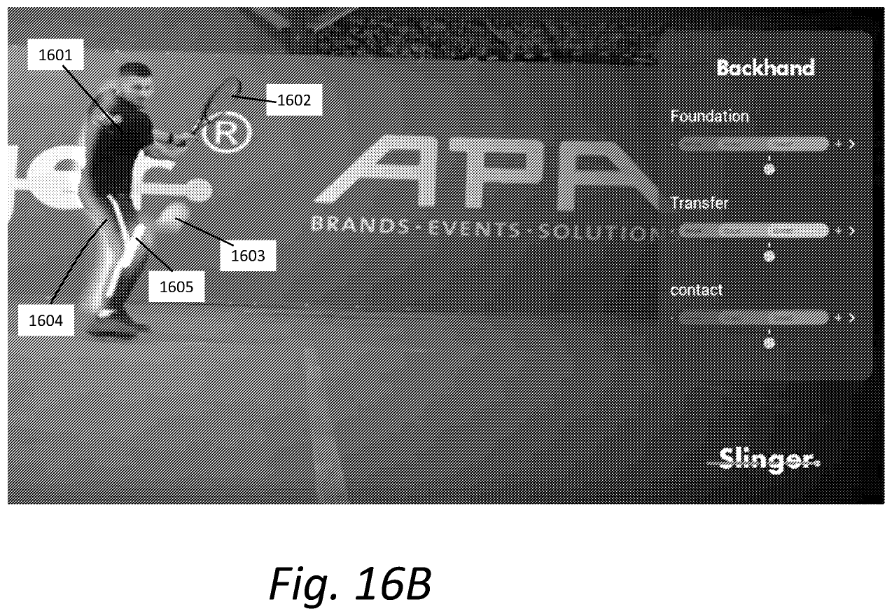

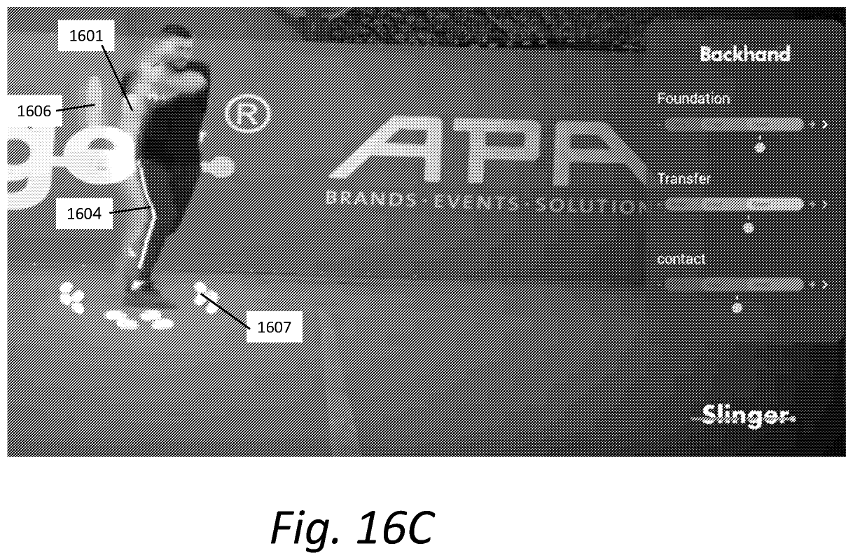

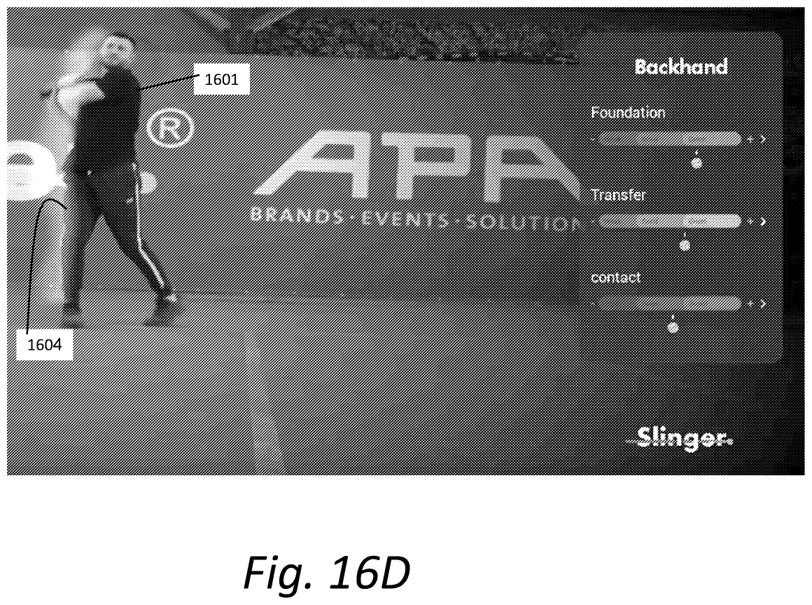

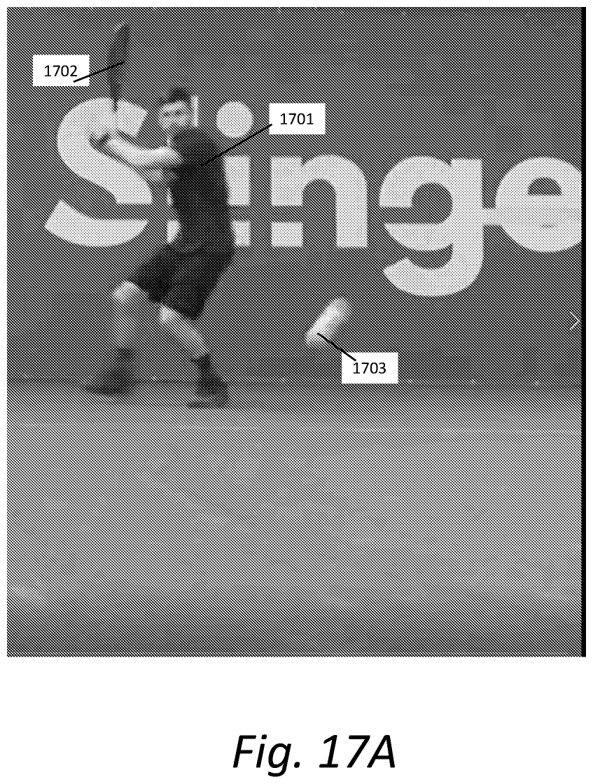

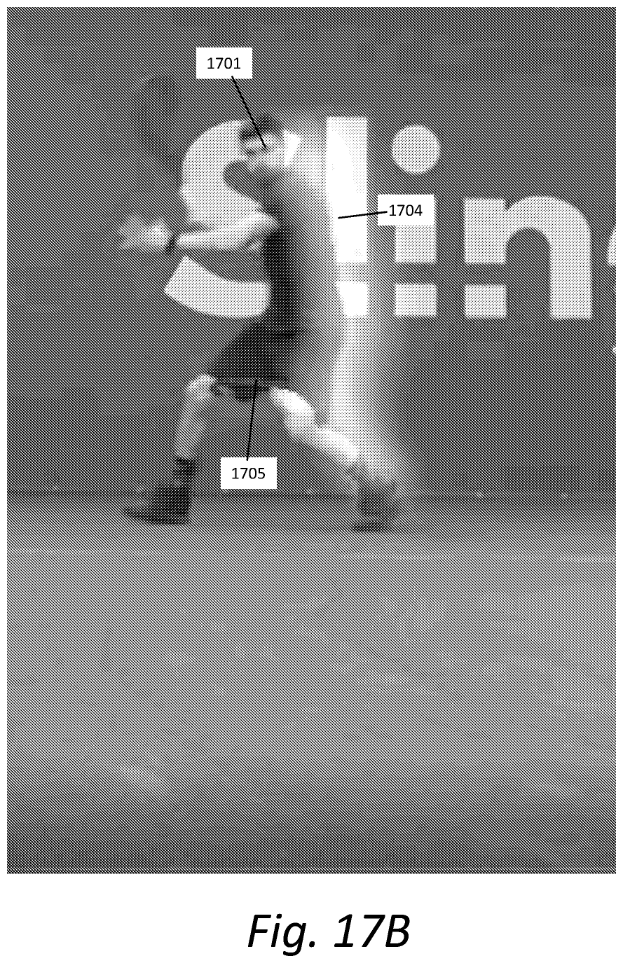

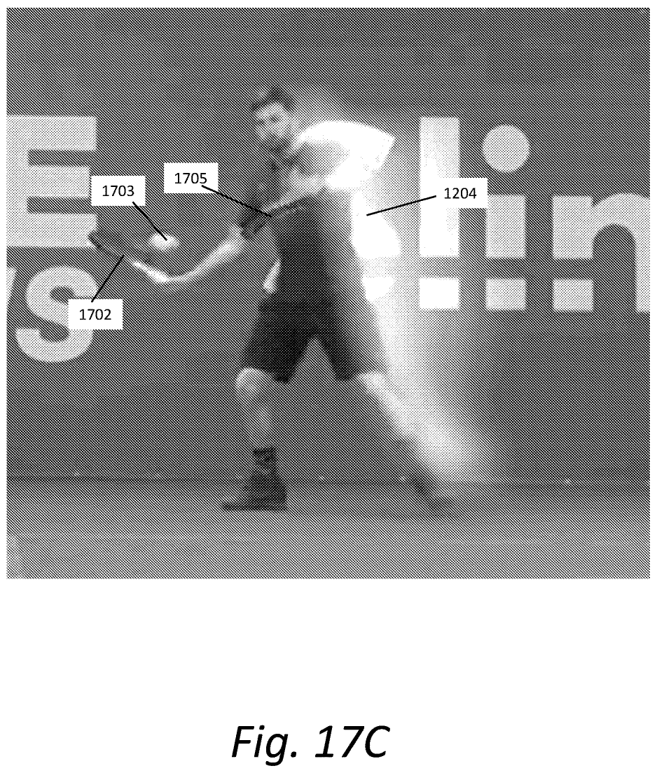

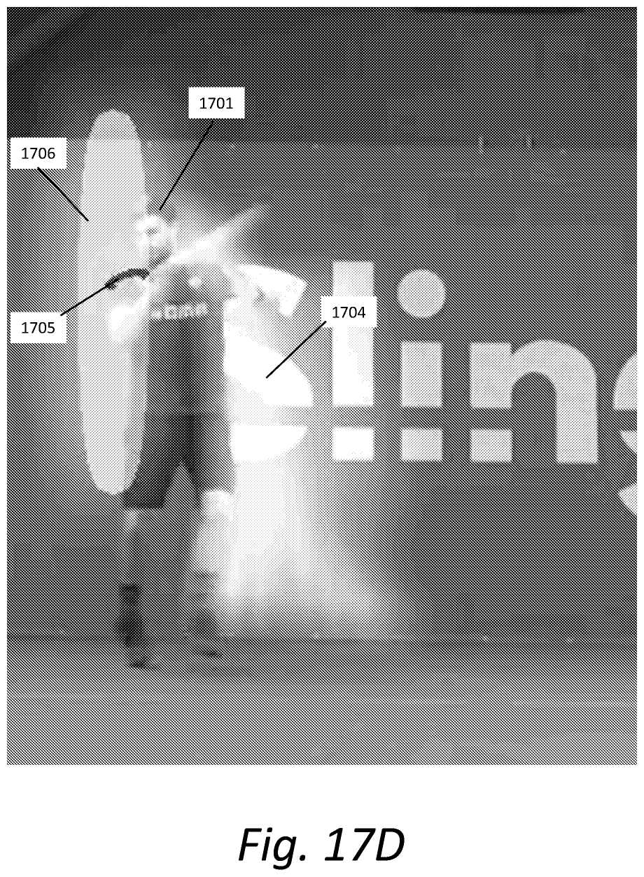

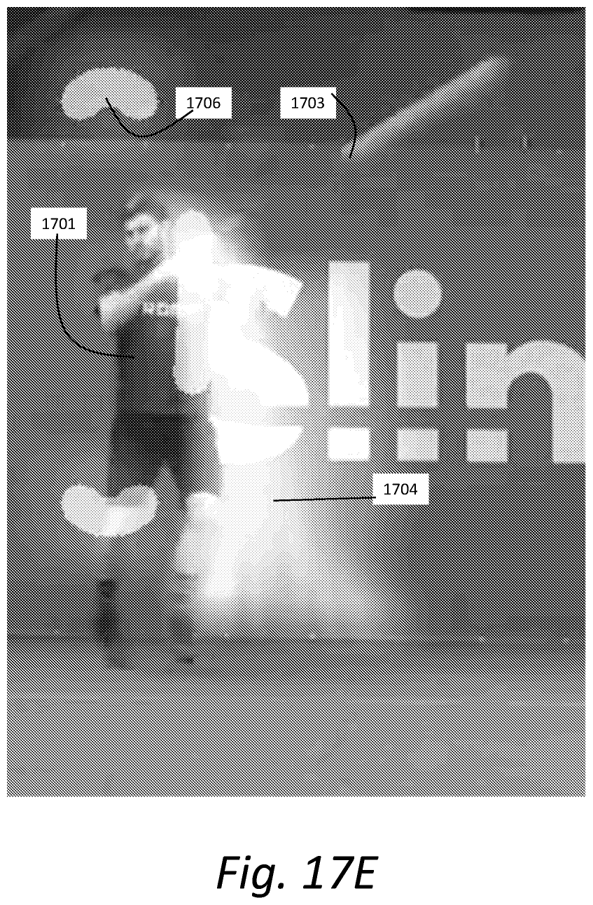

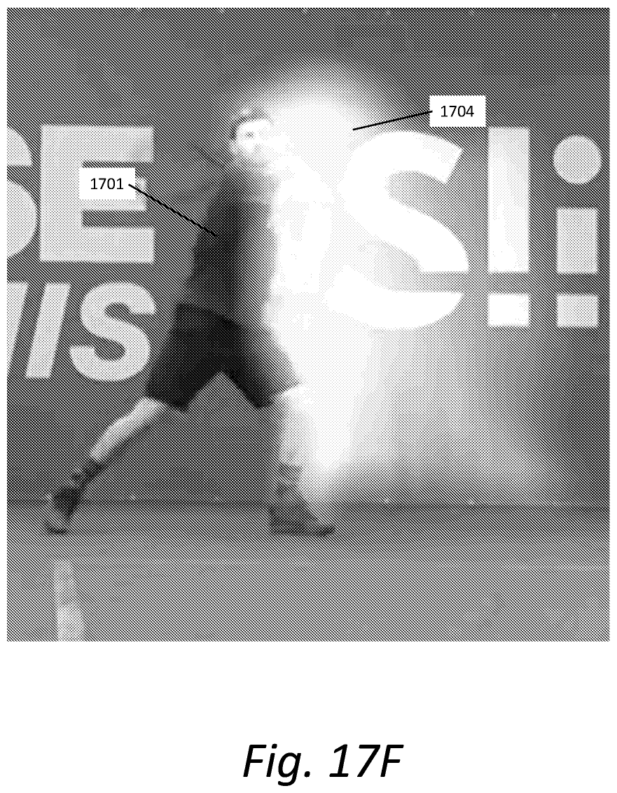

FIGS. 16A-16D depict an entire series of frames of a visual display overlaid on a backhand shot, and FIGS. 17A-17F depict a series of frames of a visual display overlaid on a forehand shot. Referring to FIG. 16A, player 1601 is shown holding racket 1602, prepared to take a shot. The background is brightly colored. In FIG. 16B, the ball 1603 is approaching the racket. Visual overlay 1604 indicates the beginning of the weight transfer, and visual overlay 1605 draws the user's attention to the foundation. The background has shifted to black and white. In FIG. 16C, the player 1601 is contacting the ball. The contact point is indicated with ellipse 1606. The weight transfer gradient fade 1604 is fully established, and the foundation display has turned into an explosion from the feet 1607. At FIG. 16D, after the completion of the shot, the weight transfer gradient fade 1604 remains even after the other visual overlays have been removed from the display.

Throughout the playback, the table is present on the right side, showing the scales for quality of foundation, transfer, and contact point. When the playback shows a sequence of shots, the table initially displays the scales for a previous shot. After completion of a new shot, the table updates to show the values for the new shot. Thus, in FIG. 16A, the table shows scales of the previous shot, whereas in FIG. 16D, the table shows values for the new shot.

In FIG. 17A, player 1701 is holding racket 1702 and is preparing to strike ball 1703. In FIG. 17B, a weight transfer gradient 1704 begins to be shown on the player 1701. In addition, ring 1705, representing flow of power, begins to travel up the player's body. At FIG. 17C, the user is close to striking ball 1703 with racket 1702. The power flow ring 1705 has traveled further up the body, and the weight transfer gradient fade 1704 is wider. FIG. 17D represents the visual overlays immediately after contact with the ball. A contact point ellipse 1706 is formed, the weight transfer gradient fade 1704 is even wider, and the power flow ring 1705 reaches the shoulder. At FIG. 17E, the ellipse 1706 representing contact point has “exploded.” At FIG. 17F, once again, the gradient fade 1704 is retained after completion of the playback of the shot.

Although, in the illustrated embodiments, the pictorial overlay is overlaid on a video stream of the ball strike, the visual display may also be overlaid on a photo, an animated avatar of the player, or any combination thereof.

The system and method described above may be used to evaluate other execution parameters, in addition to those set forth above. For example, in tennis, the swing line refers to the shape taken by the arm as a player strikes the ball. Examples of swing lines include Eastern Forehand, Semi Western Forehand, Western Forehand, Eastern or Southwestern Backhand, Eastern Backhand, Continental Backhand, and Semi-Western Backhand. Smartphone-based artificial intelligence is currently unable to directly detect specific grips in tennis. However, it is possible to detect and categorize the swing line, based on different appearances of the pose points during the shot. As a result, analysis of the pose points may also provide output regarding the swing line and, consequently, the tennis grip.

Likewise, different tennis shots feature different leg stances. Examples of common leg stances include neutral stance, closed stance, semi open stance, and semi-closed stance. The artificial intelligence of the pose points may thus include identification and analysis of the leg stance relative to the best matched shot.

### Non-Sports Applications

The system described herein is built on the fundamentals of bio-mechanics and physics. Sports provides a structure for analysis of a certain kinematic event. However, the core system can also be displayed in its own respect. For example, the Base of support, Center of Gravity, Line of Gravity, Ratio of Feet separation-shoulder width, load bearing limb, c-factor(hip-shoulder), c-factor(shoulder-ankle), v-factor etc. are all relevant to non-sports movements as well. The system represents the basic movement of the human anatomy as understood with science. These movements may be quantified by the classic mechanical constraints that they follow or breach. Since the system's building blocks are so fundamental, the system may be used for non-structured environments other than sports. Users may selectively choose an execution metric that is either in the core layer (i.e., analysis of body movements irrespective of the sports that are played) or the sport layer to analyze it over time. The system may display feedback regarding the physical activity through the pose points, as discussed, or through a three-dimensional avatar. The three-dimensional avatar may have different portions of the body colored differently, in order to emphasize certain aspects of the movement or imbalances.

1-49. (canceled)

50. A method of performing kinematic analysis of a ball-striking event, comprising:
receiving a video of a player during a ball-striking event containing a plurality of ball strikes;
performing pose estimation on images of the player in multiple frames within the video, and deriving pose points of the player in each frame, wherein said pose points correspond to joints of the player and connections between adjacent pose points correspond to limbs of the player;
converting positions of pose points of the player into signals on an X-Y-Z plot, based on positions of said joints and limbs of the player in each two-dimensional frame from the multiple frames, and wherein X represents lateral movement, Y represents up-and-down movement, and Z represents front-and-back movement;
determining, based on said signals, a series of frames within the video in which a ball-strike occurs,
for each series of frames in which a ball-strike occurs, orienting a view of the pose points in order to enable visual assessment of one or more execution parameters associated with kinematic performance of the player during one or more ball strikes within the ball-striking event; and
displaying a visual representation of the one or more execution parameters overlaid on the oriented view of the pose points during the ball-striking event.

51. The method of claim 50, wherein the determining step comprises:
determining frames of interest based on at least one of lateral movement of the pose points within the X-direction and front-and-back movement in the Z direction; and
within the frames of interest, determining existence of a shot based on up-and-down movement of the pose points.

52. The method of claim 50, further comprising one of the following:
classifying a ball strike as a type of shot based on a comparison of pose point signals of the player during the ball strike to pose point signals of the player or different players in previously-classified ball-striking events;
generating a time series of pose points corresponding to different stages of a ball strike;
representing a kinematic chain transferred through the player's joints during the ball-striking event;
displaying a visual representation of the one or more execution parameters overlaid on a visual display representing the player during the ball-striking event

53. The method of claim 50, wherein:
the one or more ball strikes are selected from a group consisting of: a tennis shot; a table tennis shot; a golf shot; a baseball swing; or a cricket bat; or
the video is a video captured by a handheld mobile device; or
the one or more execution parameters comprises stability of a foundation of the player prior to a ball strike, wherein the foundation is measured as a ratio between a distance between the player's feet and the distance between the player's shoulders as viewed in the x-z plane; or
the one or more execution parameters comprises a pressure index prior to a ball strike, wherein the pressure index is defined as a change in a center of gravity of the player over a course of a defined period prior to a ball strike; or
the one or more execution parameters comprises a shift in foundation post-swing; or
the one or more execution parameters comprises a weight transfer of a torso and head of the player, and the method further comprises displaying a triangle overlaying an image of the torso the player during the ball strike, and changing the display of the triangle based on whether the torso of the player is predominantly behind, in front of, or parallel to an ankle line over a course of a shot; or
the one or more execution parameters comprise a hip-neck stretch, and further comprising displaying the hip-neck stretch as a distance between a mid-hip point and a neck on pose points representing the player during the ball-striking event; or
the one or more execution parameters include a symmetry of the player's body during the swing, and further comprising displaying a polygon drawn by the player's shoulder's elbows, and wrists over the pose points viewed in the transverse plane during the ball-striking event; or
the one or more visual representations include a C-factor, representing extent of rotation of the shoulder and hips relative to a frontal plane of the player, and a V-factor, representing a variation of angle of separation between a shoulder line and a hip line.

54. The method of claim 53, further comprising:
representing the C-factor as an inner curve nested within an outer curve, wherein the inner curve represents rotation of the hips, and the outer curve represents rotation of the shoulders; or
representing the V-factor as a time series of an angle of separation between the shoulder line and the hip line.

55. The method of claim 53, further comprising displaying the pressure index as a time-lapse depiction of weight transfer overlaid on a visual display representing the player during the ball-striking event.

56. The method of claim 55, wherein the time-lapse depiction comprises a set of lines, wherein each line corresponds to a single time unit.

57. The method of claim 56, wherein the set of lines is weighted in a gradient fade, in which lines corresponding to earlier time units are sequentially blurred relative to lines corresponding to later time units, or wherein the set of lines is applied to only a portion of the player's body.

58. A method of performing kinematic analysis of a ball-striking event, comprising:
receiving a video of a player during a ball-striking event containing a plurality of ball strikes;
determining a series of frames within the video in which a ball-strike occurs; and
determining a pivot frame of a ball strike within the video, in which the ball is contacted by a ball-striking implement, wherein the step of determining the pivot frame comprises:
selecting, for each of the frames in the series, signals representing pose points of body parts of the player that form an X-factor, said X-factor defined as an angle formed between said player's shoulder line and said player's waist line;
comparing the signals forming the X-factor of the ball-strike with signals forming X-factors calculated from previously recorded ball-strikes;
on the basis of said comparison, determining a best match of the ball-strike to one of the previously recorded ball strikes; and
determining the pivot frame based on a comparison to a pivot frame of the best matched ball strike.

59. The method of claim 58, wherein the step of determining the series of frames further comprises:
performing pose estimation on images of the player in multiple frames within the video;
converting positions of pose points of the player into signals on an X-Y-Z plot, wherein X represents lateral movement, Y represents up-and-down movement, and Z represents front-and-back movement;
determining frames of interest based on at least one of lateral movement of the pose points within the X-direction and front-and-back movement in the Z direction; and
determining existence of a shot based on up-and-down movement of the pose points.

60. The method of claim 58, wherein the step of determining a best match comprises performing a Euclidean distance analysis of the signals making the X-factor compared to signals making the X-factors of the previously recorded ball-strikes or further comprises performing a dynamic time warping (DTW) algorithm to the signals from the ball-strike and the signals of the previously recorded ball strikes.

61. The method of claim 58, further comprising classifying a ball-strike as a type of shot on a basis of a comparison of pose point signals of the player during the ball-strike to pose-point signals of players in previously classified ball-striking events or comparing one or more execution parameters of a ball strike to the same one or more execution parameters of the best-matched previously recorded ball strike.

62. The method of claim 61, wherein:
the one or more execution parameters includes a weight transfer of the player during the ball strike; or
the one or more execution parameters includes a foundation posture and a landing posture of the player during the ball strike; or
the one or more execution parameters includes a location of a contact point of a ball-striking implement with the ball relative to the player's body; or
the one or more execution parameters includes a swing line during the ball strike or a leg stance during the ball strike.

63. The method of claim 62, further comprising comparing a consistency of the location of the contact point with other locations of contact points during the ball-striking event.

64. The method of claim 58, wherein the one or more ball strikes are selected from the group consisting of: a tennis shot; a table tennis shot; a golf shot; a baseball swing; or a cricket bat or the video is a video captured by a handheld mobile device.

65. A method of providing feedback related to kinematic performance of a player during a ball-striking event, comprising:
comparing one or more execution parameters, associated with the kinematic performance of a player during one or more ball strikes within the ball-striking event, with the same one or more execution parameters of a previously recorded ball strikes; and
displaying one or more visual representations of the one or more execution parameters as an integrative feedback display overlaid on a visual display representing the player during the ball-striking event;
wherein the one or more execution parameters comprises weight transfer of the player during a ball strike, and the visual representation is a time-lapse depiction of the weight transfer.

66. The method of claim 65, wherein:
the time-lapse depiction comprises a set of lines, wherein each line corresponds to a single time unit; or
the one or more execution parameters further includes a foundation posture and a landing posture of the player during a ball strike, and the visual representation includes at least one of (1) a geometric shape extending between the player's feet; and (2) an animation extending from the player's knees to below the player's feet; or
the one or more execution parameters further includes a contact point of the ball with the ball striking implement, and the visual representation includes one or more lines conveying the impact point of the ball with the ball striking implement; or
the visual display representing the player is selected from a group consisting of: a photo, a video stream, an animated avatar of the player, or any combination thereof.

67. The method of claim 66, wherein the set of lines is weighted in a gradient fade, in which lines corresponding to earlier time units are sequentially blurred relative to lines corresponding to later time units or is applied to only a portion of the player's body.

68. The method of claim 65, further comprising displaying educative information with respect to improving the one or more execution parameters.

69. A system including a non-transitory computer readable medium having stored thereon software that, when executed by a processor, cause the processor to perform the following steps:
receiving a video of a player during a ball-striking event containing a plurality of ball strikes;
performing pose estimation on images of the player in multiple frames within the video, and derive pose points of the player in each frame, wherein said pose points correspond to joints of the player and connections between adjacent pose points correspond to limbs of the player;
converting positions of pose points of the player into signals on an X-Y-Z plot, based on positions of said joints and limbs of the player in each two-dimensional frame from the multiple frames, and wherein X represents lateral movement, Y represents up-and-down movement, and Z represents front-and-back movement;
determining, based on said signals, a series of frames within the video in which a ball strike occurs;
for each series of frames in which a ball strike occurs, orienting a view of the pose points in order to enable visual assessment of one or more execution parameters associated with kinematic performance of the player during one or more ball strikes within the ball striking event; and
displaying a visual representation of the one or more execution parameters overlaid on the oriented view of the pose points during the ball-striking event.

70. The system of claim 69, further comprising a display device, adapted to receive instructions for generating the visual representation from said processor, and to display the visual representation to a user thereof, wherein the computer-readable medium, processor, and the display device are integrated in a single device, being a handheld smartphone or tablet.
